(19) United States
(12) Patent Application Publication
(10) Pub. No.: US 2022/0186301 A1
(43) Pub. Date: Jun. 16, 2022

(54) METHODS AND COMPOSITIONS RELATED TO TARGET ANALYSIS

(71) Applicant: PRESIDENT AND FELLOWS OF HARVARD COLLEGE, Cambridge, MA (US)

(72) Inventors: Huy Quoc NGUYEN, Cambridge, MA (US); Shyamtanu CHATTORAJ, Cambridge, MA (US); George M. CHURCH, Cambridge, MA (US); Chao-ting WU, Cambridge, MA (US)

(73) Assignee: PRESIDENT AND FELLOWS OF HARVARD COLLEGE, Cambridge, MA (US)

(21) Appl. No.: 17/631,167

(22) PCT Filed: Jul. 30, 2020
(86) PCT No.: PCT/US2020/044151

§ 371 Date: Jan. 28, 2022

Related U.S. Application Data
(60) Provisional application No. 62/880,216, filed on Jul. 30, 2019.

(51) Int. Cl. C12Q 1/6841

(52) U.S. Cl. CPC C12Q 1/6841

(57) ABSTRACT

The technology described herein is directed to methods, systems, and compositions for analyzing, detecting, and/or visualizing target molecules. Described herein are compositions and systems comprising oligonucleotide tags comprising barcoded cassettes. Also described herein are methods for analyzing target molecules, the method comprising contacting a sample with at least one oligonucleotide tag, contacting the sample with at least two readout molecules, and detecting the relative spatial order of the readout molecules.

### CROSS-REFERENCE TO RELATED APPLICATIONS

This application claims benefit under 35 U.S.C. § 119(e) of U.S. Provisional Application No. 62/880,216 filed Jul. 30, 2019, the contents of which are incorporated herein by reference in their entirety.

### GOVERNMENT SUPPORT

This invention was made with government support under Grant Nos. GM123289 and HG008525 awarded by the National Institutes of Health. The government has certain rights in the invention.

### SEQUENCE LISTING

The instant application contains a Sequence Listing which has been submitted in ASCII format via EFS-Web and is hereby incorporated by reference in its entirety. Said ASCII copy, created on Jul. 27, 2020, is named 002806-094580_SL.txt and is 1,055 bytes in size.

### TECHNICAL FIELD

The technology described herein relates to methods, systems, and compositions for analyzing, detecting, and/or visualizing target molecules.

### BACKGROUND

Visualization of targets at the cellular and subcellular level typically makes use of fluorophores linked to detection molecules (e.g., antibodies, oligos, synthetic nucleic acids, etc.). Due to the spectral overall limitations of fluorescent microscopy, only 4-5 targets can be visualized simultaneously. However, some applications necessitate the visualize of more than 5 targets simultaneously, e.g., a complex system comprising more than 5 components or a large target molecule such as a chromosome. As such, new methods are needed that allow for the simultaneous detection of many targets or many regions of a target.

### SUMMARY

The technology described herein is directed to methods, systems, and compositions for analyzing, detecting, and/or visualizing target molecules which offer the ability to visualize more targets at one time than is possible with prior technologies. As an example, when using fluorescent labels to detect target biomolecules, the present methods cause detection probes using the 4 colors to localize to targets in groups, where the probes assemble at each different target in groups that form a barcoded sequence of colors. Use of 4 colors with existing technology would permit detection of only 4 different targets. The disclosed methods, using only a 2-bit barcode, could detect 16 targets simultaneously.

As disclosed herein, target molecules analysis can be performed with OligoCASSEQ technology, comprising an oligonucleotide tag (e.g., an Oligopaint). Readout molecules can be non-enzymatically used to detect the identity of the specific oligonucleotide tag (e.g., an Oligopaint).

In one aspect, described herein is a method of analyzing at least one target molecule in a sample, the method comprising: (a) contacting the sample with at least one oligonucleotide tag, each oligonucleotide tag comprising: (i) a recognition domain that binds specifically to a target molecule to be analyzed, and (ii) at least one street comprising at least one cassette, each cassette comprising: (1) a barcode region comprising at least 1 nucleotide, flanked on at least one side by an anchor region; wherein each oligonucleotide tag's street is unique from the streets of the other oligonucleotide tags of step (a) at least in that A. the spatial order of the cassettes within the street differs or B. that the sequence of the barcode region differs from the barcode regions of the other oligonucleotide tags of step (a); (b) contacting the sample with at least two readout molecules, wherein each readout molecule comprises: (i) an oligonucleotide that hybridizes specifically with a cassette of at least one oligonucleotide tag used in step (a); and (ii) a detection molecule; wherein the at least two readout molecules collectively comprise at least two distinguishable detection molecules; and (c) detecting the relative spatial order of the detection molecules hybridized to at least one oligonucleotide tag, wherein the at least one oligonucleotide tag is hybridized to the at least one target molecule, whereby the relative spatial order of the detection molecules permits identification of which oligonucleotide tag is hybridized to the target molecule at that location.

In some embodiments of any of the aspects, the barcode region comprises 1-10 nucleotides.

In some embodiments of any of the aspects, the street comprises at least 3 cassettes.

In some embodiments of any of the aspects, the barcode region is flanked on each side by an anchor region.

In some embodiments of any of the aspects, the anchor regions of all of the oligonucleotide tags are constant.

In some embodiments of any of the aspects, the specific hybridization of a readout molecule to a cassette is determined by the identity of the barcode region.

In some embodiments of any of the aspects, the detection molecule is a fluorophore.

In some embodiments of any of the aspects, the detecting is performed with fluorescence microscopy.

In some embodiments of any of the aspects, the detection molecule comprises biotin, amines, metals, anchoring molecules, or acrydite.

In some embodiments of any of the aspects, the detecting is performed with at least single cell resolution.

In some embodiments of any of the aspects, step (b) comprises contacting the sample with at least 4 readout molecules.

In some embodiments of any of the aspects, step (b) comprises contacting the sample with a group of readout molecules that collectively comprise at least 3 distinguishable detection molecules.

In some embodiments of any of the aspects, step (b) comprises contacting the sample with a group of readout molecules that collectively comprise at least 4 distinguishable detection molecules.

In some embodiments of any of the aspects, at least 2 target molecules are analyzed concurrently.

In some embodiments of any of the aspects, at least 3 target molecules are analyzed concurrently.

In some embodiments of any of the aspects, at least 10 target molecules are analyzed concurrently.

In some embodiments of any of the aspects, at least 20 target molecules are analyzed concurrently.

In some embodiments of any of the aspects, the target molecule is a nucleic acid, a polypeptide, a cell surface molecule, or an inorganic material.

In some embodiments of any of the aspects, the target molecule is a DNA or mRNA. In some embodiments of any of the aspects, the sample is a cell, cell culture, or tissue sample.

In another aspect described herein is a system for analyzing at least one target molecule in a sample, the system comprising: (a) a detector that can detect at least two detectable molecules; (b) at least one oligonucleotide tag, each oligonucleotide tag comprising: (i) a recognition domain that binds specifically to a target molecule to be analyzed, and (ii) a street comprising at least one cassette, each cassette comprising: (1) a barcode region comprising at least 1 nucleotide, flanked on at least one side by an anchor region; wherein each oligonucleotide tag's street is unique from the streets of the other oligonucleotide tags of (b) at least in that A. the spatial order of the cassettes within the street differs or B. that the sequence of the barcode region differs from the barcode regions of the other oligonucleotide tags of (b); and (c) at least two readout molecules, wherein each readout molecule comprises: (i) an oligonucleotide that hybridizes specifically with a cassette of at least one oligonucleotide tag used in (b); and (ii) a detection molecule; wherein the at least two readout molecules collectively comprise at least two distinguishable detection molecules; and wherein a sample is contacted with the at least one oligonucleotide tag and the at least two readout molecules, and the relative spatial order of the detection molecules hybridized to at least one oligonucleotide tag is detected, wherein the at least one oligonucleotide tag is hybridized to the at least one target molecule, whereby the relative spatial order of the detection molecules permits identification of which oligonucleotide tag is hybridized to the target molecule at that location.

### BRIEF DESCRIPTION OF THE DRAWINGS

FIG. 1A-FIG. 1H are a series of images and schematics showing multiplexed genomic tracing with OligoCASSEQ. FIG. 1A is a schematic showing modified Oligopaints for OligoCASSEQ. The genomic homology region hybridizes to genomic DNA and is flanked by the non-genomic homology regions, the MainStreet and the BackStreet. Streets contain universal priming regions that allow for amplification of the Oligopaints library. Streets also contain Sequencing Cassettes (e.g., Cassette 1, Cassette 2, Cassette 3, and Cassette 4) that are used to multiplex. Four cassettes are depicted here, but the total number of cassettes is unlimited.

FIG. 1B is a schematic showing the sequencing cassette from FIG. 1A. The barcode region contains a variable region (X). Each bit (X) can be coded by one of four nucleotides (e.g., A, C, T, or G). A 5 nucleotide (nt) barcode is shown here, but the barcode can vary in length. Increasing the barcode length increases multiplexing. Barcodes are interrogated by specific Readout Oligos (see e.g., FIG. 1C). Each probe contains different barcodes, which enables multiplexing. On each side of the barcode regions are the anchor regions (e.g., 5 nt shown here, but the barcode region length can vary) that are constant between all probes. Anchor regions provide stable hybridization and allow for barcodes to be differentiated.

FIG. 1C is a schematic showing example Readout Oligos for 2 positions within a cassette. Each pool of Readout Oligos are used to interrogate specific barcode positions. Fluor-labeled Readout Oligos base pair specifically to unique bases (e.g., A, C, G, and T) at specific positions and contain a mixture of nucleotides at non-interrogation bases (“N”). The anchor (“Anch”) nucleotide sequence is cassette specific (e.g., cassette 1 shares specific anchors, and cassette 2 shares different anchors).

FIG. 1D is a schematic showing an example OligoCASSEQ experimental workflow to read out Cassette positions 1 and 2. From left to right, Oligopaint probes are first hybridized to genomic DNA. A specific pool of Readout Oligos are hybridized to sequence barcode position 1 on cassette 1 (“A” on Cassette). The red “T” Readout Oligo binds with complete complementarity to the Cassette. After hybridization, an image is taken, and the readout Oligos are washed out with 60% formamide in 2× SSCT. Position 2 on Cassette 1 can then be interrogated by hybridizing the position 2 Cassette 1 oligos. Other positions and cassettes are interrogated in the same way.

FIG. 1E-FIG. 1H are a series of images showing OligoCASSEQ localization of five loci along human Chromosome 2 (Chr.2). FIG. 1E is a schematic showing Chr.2 loci, the nucleotide barcode, and the color barcode used for the demonstration experiments (see e.g., FIG. 1F-FIG. 1H).

FIG. 1F is an image showing micrographs of OligoCASSEQ interrogation of two barcode positions (see e.g., the top and middle sections), followed by re-interrogation of the—1st position in the same nucleus (see e.g., the bottom section). The schematic above each micrograph displays the color code of specific loci at different barcode positions. If using both red and cyan labeled probes, note that red and cyan colors overlap due to suboptimal microscope filter sets. However, cyan is brighter than red when red and cyan do overlap. Micrographs are maximum intensity z-projections from multiple z-slices. The cells are PGP1-F cells.

FIG. 1G is an image showing barcode identification. After sequencing of at least 2 positions, the OligoCASSEQ foci can be decoded.

FIG. 1H is an image showing that OligoCASSEQ allows chromosome tracing, as the genomic location of every focus can be identified. The line is a 2D trace based on the decoded foci. The arrowhead marks the bottom of the chromosome (e.g., the locus closest to telomere of the long, q, arm). OligoCASSEQ can also be used for super-resolution microscopy by using readout probes (see e.g., FIG. 1C) containing compatible fluorophores.

### DETAILED DESCRIPTION

Embodiments of the technology described herein comprise methods of analyzing a target molecule using OligoCASSEQ. As described herein, OligoCASSEQ refers to methods, systems, and/or compositions comprising an oligonucleotide tag (e.g., an Oligopaint). The disclosed methods permit the visualization of at least two targets or at least two regions of one target simultaneously. Furthermore, through the use of barcoded cassettes, the disclosed methods can permit the visualization of greater than 4 targets simultaneously, more than can currently be detected with traditional Oligopaints.

These methods are superior to alternative methods. The disclosed methods do not require enzymes such as ligases or polymerases, which are required by alternative methods. The disclosed methods also involve shorter oligos and fewer oligos than are required for alternative techniques. Finally, the disclosed methods are simpler and less expensive than the alternatives.

Accordingly, in one aspect described herein is a method of analyzing at least one target molecule in a sample, the method comprising: (a) contacting the sample with at least one oligonucleotide tag (e.g., an Oligopaint), each oligonucleotide tag comprising: (i) a recognition domain that binds specifically to a target molecule to be analyzed, and (ii) at least one street comprising at least one cassette, each cassette comprising: (1) a barcode region comprising at least 1 nucleotide, flanked on at least one side by an anchor region; wherein each oligonucleotide tag's street is unique from the streets of the other oligonucleotide tag of step (a) at least in that the spatial order of the cassettes within the street differs; (b) contacting the sample with at least two readout molecules, wherein each readout molecule comprises: (i) an oligonucleotide that hybridizes specifically with a cassette of at least one oligonucleotide tag used in step (a); and (ii) a detection molecule; wherein the at least two readout molecules collectively comprise at least two distinguishable detection molecules; and (c) detecting the relative spatial order of the detection molecules hybridized to at least one oligonucleotide tag, wherein the at least one oligonucleotide tag is hybridized to the at least one target molecule, whereby the relative spatial order of the detection molecules permits identification of which oligonucleotide tag is hybridized to the target molecule at that location.

In other words, OligoCASSEQ methods can comprise contacting a sample with an oligonucleotide tag (e.g., an Oligopaint), contacting the sample with at least two readout molecules, and determining the spatial order of the barcoded cassettes.

In another aspect described herein is a system for analyzing at least one target molecule in a sample, the system comprising: (a) a detector that can detect at least two detectable molecules; (b) at least one oligonucleotide tag (e.g., an Oligopaint), each oligonucleotide tag comprising: (i) a recognition domain that binds specifically to a target molecule to be analyzed, and (ii) a street comprising at least one cassette, each cassette comprising: a barcode region comprising at least 1 nucleotide, flanked on at least one side by an anchor region; wherein each oligonucleotide tag's street is unique from the streets of the other oligonucleotide tags of (b) at least in that A. the spatial order of the cassettes within the street differs or B. that the sequence of the barcode region differs from the barcode regions of the other oligonucleotide tags of (b); and (c) at least two readout molecules, wherein each readout molecule comprises: (i) an oligonucleotide that hybridizes specifically with a cassette of at least one oligonucleotide tag used in (b); and (ii) a detection molecule; wherein the at least two readout molecules collectively comprise at least two distinguishable detection molecules. In some embodiments of any of the aspects, a sample is contacted with the at least one oligonucleotide tag and the at least two readout molecules, and the relative spatial order of the detection molecules hybridized to at least one oligonucleotide tag is detected (e.g., with the detector), wherein the at least one oligonucleotide tag is hybridized to the at least one target molecule, whereby the relative spatial order of the detection molecules permits identification of which oligonucleotide tag is hybridized to the target molecule at that location. In some embodiments of any of the aspects, detection data is outputted on a display.

As used herein, the term “oligonucleotide tag” is an oligonucleotide that comprises a recognition domain and/or at least one street. In some embodiments of any of the aspects, the oligonucleotide tag comprises or is comprised by oligonucleotides including but not limited to Oligopaints, multiplexed error-robust fluorescence in situ hybridization (MERFISH) oligos, seqFISH oligos, RNA sequential probing of targets (SPOTs) oligos, high-coverage microscopy-based technology (Hi-M) oligos, or optical reconstruction of chromatin architecture (ORCA) oligos or any to oligonucleotide used for FISH methods and/or any oligonucleotides that has a sequence complementary (e.g. recognition domain) to a target molecule, e.g., an oligonucleotide sequence, a portion of a DNA sequence, or a particular chromosome or sub-chromosomal region of a particular chromosome. For further details, see e.g., Cardozo et al., Mol Cell. 2019 Apr. 4; 74(1):212-222; Mateo et al., Nature. 2019 April; 568(7750):49-54; Wang et al., Scientific Reports volume 8, Article number: 4847 (2018); Shah et al., Neuron, Volume 92, Issue 2, 19 Oct. 2016, Pages 342-357; Eng et al., Nat Methods. 2017 December; 14(12):1153-1155; each of which is incorporated herein by reference in its entirety.

As used herein, the term “street” refers to a portion of the oligonucleotide tag (e.g., an Oligopaint) that does not have identity with a target sequence or does not hybridize to a target sequence. As used herein, “cassette” refers to a region of the street that comprises a barcode region and at least one anchor region. As used herein, “barcode region” refers to a region of a cassette comprising at least 1 nucleotide. As used herein, “anchor region” refers to a region of the cassette refers to a region of a cassette that is specific and/or constant to a set of cassettes and/or is complementary to the anchor-hybridizing region of at least one readout molecule. As used herein, “readout molecule” refers to a molecule comprising 1) a detection molecule or moiety and 2) an oligonucleotide sequence that is complementary to at least a portion of at least one cassette of at least one oligonucleotide tag (e.g., an Oligopaint) and/or hybridizes specifically with at least a portion of least one cassette of at least one oligonucleotide tag (e.g., an Oligopaint).

In some embodiments of any of the aspects, the oligonucleotide tag (e.g., an Oligopaint) comprises at least one street, and said street comprises at least one cassette. As used herein, “cassette” refers to a region of the street that comprises a barcode region and at least one anchor region. As a non-limiting example, the street comprises 1 cassette, 2 cassettes, 3 cassettes, 4 cassettes, 5 cassettes, 6 cassettes, 7 cassettes, 8 cassettes, 9 cassettes, or at least 10 cassettes.

In some embodiments of any of the aspects, wherein an oligonucleotide tag (e.g., an Oligopaint) comprises at least 1 cassette, a population of oligonucleotide tags comprises subpopulations of oligonucleotide tags, wherein each subpopulation is defined by the type of cassette(s) present in the oligonucleotide tag. Each cassette type comprises at least one unique and/or distinguishable anchor region (i.e., the anchor region can be cassette type-specific), and each individual cassette comprises a unique and/or distinguishable barcode region. As a non-limiting example, a population of oligonucleotide tags (e.g., an Oligopaint) comprises a Type 1 cassette and a Type 2 cassette, wherein the set of Type 1 cassettes share at least one anchor region that is different from the anchor region(s) shared by the set of Type 2 cassettes. As a non-limiting example, a Type 1 cassette comprises anchor region A and anchor region B, and a Type 2 cassette comprises anchor region C and anchor region D. In some embodiments of any of the aspects, the cassette subpopulations can share anchor regions, as long at least one anchor region is unique to and/or distinguishable for each cassette subpopulation. As a non-limiting example, a Type 1 cassette comprises anchor region A and anchor region B, and a Type 2 cassette comprises anchor region A and anchor region C.

In some embodiments of any of the aspects, an oligonucleotide tag (e.g., an Oligopaint) comprises multiple cassettes, wherein each cassette is a different type (e.g., oligonucleotide tag X comprises a Type 1 cassette, a Type 2 cassette, and a Type 3 cassette). In some embodiments of any of the aspects, an oligonucleotide tag (e.g., an Oligopaint) comprises multiple cassettes, wherein at least two cassettes are the same type (e.g., oligonucleotide tag Y comprises two Type 1 cassettes, and one Type 2 cassette). The presence of multiple copies of the same cassette type in an oligonucleotide tag (e.g., an Oligopaint) can be used to amplify the signal (e.g., recruit two readout molecules to the oligonucleotide tag). In some embodiments of any of the aspects, an oligonucleotide tag (e.g., an Oligopaint) or a population of oligonucleotide tags comprises 1 type of cassettes, 2 types of cassettes, 3 types of cassettes, 4 types of cassettes, 5 types of cassettes, 6 types of cassettes, 7 types of cassettes, 8 types of cassettes, 9 types of cassettes, or at least 10 types of cassettes. In some embodiments of any of the aspects, each type of cassette can encode a specific identifier (e.g., chromosome number, chromosome arm, chromosome region, gene, etc.).

In some embodiments of any of the aspects, each oligonucleotide tag (e.g., an Oligopaint) in a set of oligonucleotide tags has a unique arrangement of cassettes, e.g., the sequences of the cassettes may be unique, but in each different oligonucleotide tag the cassettes are arranged in a different spatial order within the street. Accordingly, in some embodiments of any of the aspects, each oligonucleotide tag (e.g., an Oligopaint) in a set of oligonucleotide tags has a unique street, e.g., at least in that the spatial order of the cassettes in each oligonucleotide tag's street differs. In some embodiments of any of the aspects, each oligonucleotide tag (e.g., an Oligopaint) in a set of oligonucleotide tags have a unique cassette or set of cassettes. In some embodiments of any of the aspects, all of the cassettes in a street are identical to each other. In some embodiments of any of the aspects, all of the cassettes in an oligonucleotide tag (e.g., an Oligopaint) are identical to each other. In some embodiments of any of the aspects, at least one cassette is different from the other cassettes in the same street or oligonucleotide tag (e.g., an Oligopaint). In some embodiments of any of the aspects, each and every cassette is different from the other cassettes in the same street or oligonucleotide tag (e.g., an Oligopaint). In some embodiments of any of the aspects, the Mainstreet comprises a cassette or a set of cassettes, and the Backstreet comprises a different cassette or a different set of cassettes. In some embodiments of any of the aspects, the Mainstreet and Backstreet share the same cassette or set of cassettes. In some embodiments of any of the aspects, each oligonucleotide tag (e.g., an Oligopaint) in a set of oligonucleotide tags, share the same cassette or set of cassettes, wherein in a set of oligonucleotide tags comprises oligonucleotide tags that hybridize to different target molecules.

In some embodiments of any of the aspects, the cassette comprises a barcode region. As used herein, “barcode region” refers to a region of a cassette comprising at least 1 nucleotide. As a non-limiting example, the barcode region comprises 1 nucleotide, 2 nucleotides, 3 nucleotides, 4 nucleotides, 5 nucleotides, 6 nucleotides, 7 nucleotides, 8 nucleotides, 9 nucleotides, or 10 nucleotides. In some embodiments of any of the aspects, at least one nucleotide of the barcode region comprises a modified nucleobase base, as described further herein.

In some embodiments of any of the aspects, the sequence of the barcode region differs from the barcode regions of the other oligonucleotide tags (e.g., an Oligopaint). In some embodiments of any of the aspects, each and every cassette has a different barcode region, e.g., a barcode region with a different sequence of nucleotides. In some embodiments of any of the aspects, each and every cassette in a street has a different barcode region than the other cassettes in the street, e.g., a barcode region with a different sequence of nucleotides. In some embodiments of any of the aspects, the sequence of the barcode region is the same and shared with at least one barcode region of the other oligonucleotide tags (e.g., an Oligopaint). In some embodiments of any of the aspects, a barcode region is flanked on at least one side by an anchor region. In some embodiments of any of the aspects, a barcode region is flanked on both sides by an anchor region.

In some embodiments of any of the aspects, a cassette comprises at least one anchor region. As used herein, “anchor region” refers to a region of the cassette that is specific and/or constant to a set of cassettes and/or is complementary to the anchor-hybridizing region of at least one readout molecule. In some embodiments of any of the aspects, each cassette comprises at least one anchor region that is unique from the at least one anchor region of all other cassettes. As a non-limiting example, the anchor region comprises at least 1 nucleotide. As a non-limiting example, the anchor region comprises 1 nucleotide, 2 nucleotides, 3 nucleotides, 4 nucleotides, 5 nucleotides, 6 nucleotides, 7 nucleotides, 8 nucleotides, 9 nucleotides, or 10 nucleotides. In some embodiments of any of the aspects, the anchor region comprises at least 5 nucleotides.

In some embodiments of any of the aspects, an anchor region comprising 5 or fewer nucleotides (e.g., 1, 2, 3, 4, or 5 nucleotides) allows for transient binding of the readout molecule to the oligonucleotide tag (e.g., an Oligopaint). As used herein, the term “transient binding” refers to weak, reversible, and/or temporary, specific interactions between molecules, i.e., readout molecules with a binding affinity such that they can bind and unbind repeatedly. Such binding affinity can be measured using a dissociation constant, Kd. In some embodiments of any of the aspects, transient binding can be defined as having a Kd in the μM range. In some embodiments of any of the aspects, an anchor region with low binding affinity to a target molecule can be used for methods wherein transient binding to the target molecule is preferred (e.g., DNA-Exchange PAINT, see e.g., US 2016/0161472 A1, Jungmann et al., Nano Lett. 2010, 10, 11, 4756-4761, each of which is incorporated by reference herein in its entirety).

Non-limiting examples of anchor regions include the following: GTCTC (SEQ ID NO: 1); CACTA (SEQ ID NO: 2); GCCCG (SEQ ID NO: 3); and TGTGC (SEQ ID NO: 4).

As a non-limiting example, the cassette comprises one anchor region or two anchor regions. In some embodiments of any of the aspects, the anchor region is 5′ to the barcode region, and/or the anchor region is 3′ to the barcode region. In some embodiments of any of the aspects, the barcode region is flanked on each side by an anchor region, e.g., one anchor region is 5′ to the barcode region, and one anchor region is 3′ to the barcode region.

Any individual cassette can optionally comprise additional sequences, e.g., linker or spacer sequences to situate it at a desired distance from other cassettes or elements of an oligonucleotide tag (e.g., an Oligopaint).

In some embodiments of any of the aspects, each oligonucleotide tag's (e.g., an Oligopaint) street is unique from the streets of the other oligonucleotide tags due to its cassette or set of cassettes. In some embodiments of any of the aspects, each oligonucleotide tag's (e.g., an Oligopaint) street is unique from the streets of the other oligonucleotide tags at least in that the spatial order of the cassettes within the street differs. As a non-limiting example, a street comprising three cassettes (e.g., cassettes “A”, “B”, and “C”) in the spatial order 5′-A-B-C-3′ has a unique spatial order of cassettes that differs compared to any other streets comprising a spatial order of cassettes selected from 5′-A-C-B-3′, 5′-B-A-C 3′, 5′-B-C-A 3′, 5′-C-A-B 3′, or 5′-C-B-A 3′, and each of the aforementioned streets are unique and differ from each other in their spatial order of cassettes.

In some embodiments of any of the aspects, each oligonucleotide tag's (e.g., an Oligopaint) street is unique from the streets of the other oligonucleotide tags at least in that the barcode region within the street differs. As a non-limiting example, a street comprising a barcode comprising for example 3 nucleotides (e.g., nucleotides “X”, “Y”, and “Z”) in the order 5′-X-Y-Z-3′ has a unique barcode that differs compared to any other streets comprising a barcode region selected from 5′-X-Z-Y-3′, 5′-Y-X-Z-3′, 5′-Y-Z-X-3′, 5′-Z-X-Y-3′, or 5′-Z-Y-X-3′, and each of the aforementioned streets are unique and differ from each other in their barcode regions.

In some embodiments of any of the aspects, each oligonucleotide tag's (e.g., an Oligopaint) street is unique from the streets of the other oligonucleotide tags at least in that the spatial order of the cassettes within the street differs, and each oligonucleotide tag's street is unique from the streets of the other oligonucleotide tags at least in that the barcode region or barcode regions within the street differs.

In some embodiments of any of the aspects, the methods describe herein comprise contacting a sample with a readout molecule. As used herein, “readout molecule” refers to a molecule comprising 1) a detection molecule or moiety and 2) an oligonucleotide sequence that is complementary to at least a portion of at least one cassette of at least one oligonucleotide tag (e.g., an Oligopaint) and/or hybridizes specifically with at least a portion of least one cassette of at least one oligonucleotide tag (e.g., an Oligopaint). As a non-limiting example, the readout molecule comprises at least one region that is complementary and hybridizes specifically to the anchor region of a cassette, e.g., an “anchor-hybridizing region”. As a non-limiting example, the readout molecule comprises at least one region that is complementary and hybridizes specifically to the barcode region of a cassette, e.g., a “barcode-hybridizing region”. As a non-limiting example, the readout molecule comprises one anchor-hybridizing region and/or two anchor-hybridizing regions. In some embodiments of any of the aspects, the anchor-hybridizing region is 5′ to the barcode-hybridizing region, and/or the anchor-hybridizing region is 3′ to the barcode-hybridizing region. In some embodiments of any of the aspects, the barcode-hybridizing region is flanked on each side by an anchor-hybridizing region, e.g., one anchor-hybridizing region is 5′ to the barcode-hybridizing region, and one anchor-hybridizing region is 3′ to the barcode-hybridizing region. In some embodiments of any of the aspects, the anchor-hybridizing region can be unique to a cassette type, as described above for anchor regions unique to a cassette type.

In some embodiments of any of the aspects, the readout molecule comprises a detection molecule, e.g., an optically-detectable detection molecule. In some embodiments of any of the aspects, the detection molecule is a fluorophore, and detecting is performed with fluorescence microscopy. In some embodiments of any of the aspects, the detection molecule comprises biotin, amines, metals, anchoring molecules, or acrydite. Non-limiting examples of detection molecules, fluorophores, and detection techniques are described further herein.

In some embodiments of any of the aspects, at least two readout molecules collectively comprise at least two distinguishable detection molecules. As a non-limiting example, two readout molecules collectively comprise two distinguishable detection molecules, three readout molecules collectively comprise three distinguishable detection molecules, four readout molecules collectively comprise four distinguishable detection molecules, or at least five readout molecules collectively comprise at least five distinguishable detection molecules. In some embodiments of any of the aspects, a pool of readout molecules comprises more readout molecules than distinguishable detection molecules, e.g., the same detection molecule can be present on multiple readout molecules.

In some embodiments of any of the aspects, a detection molecule can be linked to the 5′ end of the readout molecule, a detection molecule can be linked to the 3′ end of the readout molecule, or a detection molecule can be linked to the 5′ end and the 3′ end of the readout molecule. In some embodiments of any of the aspects, the detection molecule linked to the 5′ end of the readout molecule is the same type of detection molecule as the detection molecule linked to the 3′ end of the readout molecule. In some embodiments of any of the aspects, the detection molecule linked to the 5′ end of the readout molecule is a different type of detection molecule as the detection molecule linked to the 3′ end of the readout molecule.

In some embodiments of any of the aspects, the sample is contacted with at least two readout molecules. some embodiments of any of the aspects, the sample is contacted with at least one readout molecules. As a non-limiting example, the sample is contacted with 1 readout molecule, 2 readout molecules, 3 readout molecules, 4 readout molecules, or at least 5 readout molecules.

In some embodiments of any of the aspects, the method comprises a step of detecting the detection molecules, e.g., the detection molecules of the readout molecules hybridized to oligonucleotide tags (e.g., an Oligopaint). In some embodiments of any of the aspects, the method comprises a step of detecting the detection molecules, e.g., the detection molecules of the readout molecules hybridized to oligonucleotide tags (e.g., an Oligopaint) which are in turn hybridized to one or more targets.

In some embodiments of any of the aspects, the detecting step comprises detecting the relative spatial order of the detection molecules hybridized to the at least one oligonucleotide tag (e.g., an Oligopaint). The different labels (e.g., colors) of the readout molecules correlate with one or more cassettes, and the spatial order of the different readout molecules provides information about the order of the cassettes on a single oligonucleotide tag (e.g., an Oligopaint), allowing a large number of different oligonucleotide tags to be distinguished by the barcoded signals provided by groups of readout molecules hybridized to a single street. Accordingly, in some embodiments of any of the aspects, the relative spatial order of the detection molecules permits identification of which oligonucleotide tag (e.g., an Oligopaint) is hybridized to the target molecule at that location.

In some embodiments of any of the aspects, a detecting step comprises detecting the relative spatial order of the detection molecules of the readout molecules hybridized to the at least one cassette, whereby the relative spatial order of the detection molecules permits identification of which oligonucleotide tag (e.g., an Oligopaint) is hybridized to the target molecule at that location.

In some embodiments of any of the aspects, a detecting step comprises detecting the relative spatial order of the readout molecules hybridized to the at least one cassette, whereby the relative spatial order of the readout molecules permits identification of which oligonucleotide tag (e.g., an Oligopaint) is hybridized to the target molecule at that location.

In some embodiments of any of the aspects, the sample is contacted with a pool of readout molecules, e.g., a “readout pool”. In some embodiments of any of the aspects, the readout pool comprises readout molecules for the same cassette type (e.g., readout molecules sharing at least one unique anchor-hybridizing region). In some embodiments of any of the aspects, the readout pool comprises readout molecules for at least 1, at least 2, at least 3, at least 4, or at least 5 cassette types.

In some embodiments of any of the aspects, each readout pool is directed at determining the identity of a nucleotide at a specific position in the barcode region of a set of oligonucleotide tags (e.g., Oligopaints with a specific cassette type). As a non-limiting example, the sample is sequentially or simultaneously contacted with at least two readout pools to detect at least one nucleotide of the barcode region. As a non-limiting example, the sample is sequentially contacted with at least two readout pools to detect at first and second cassette position in one or more streets. In some embodiments of any of the aspects, the readout pool comprises 2 readout molecules, 3 readout molecules, 4 readout molecules, or at least 5 readout molecules. In some embodiments of any of the aspects, the readout pool comprises 2 distinct detection molecules, 3 distinct detection molecules, 4 distinct detection molecules, or at least 5 distinct detection molecules.

In some embodiments of any of the aspects, the readout pool comprises a set of readout molecules linked to the same type of detection molecule. As a non-limiting example, the set of readout molecules linked to the same type of detection molecule comprise the same nucleotide at one position of the barcode-hybridizing region and at least one of: (1) different nucleotides at the other positions of the barcode-hybridizing region, (2) the set is degenerate at the other positions of the barcode-hybridizing region, or (3) universal nucleotides at the other positions of the barcode-hybridizing region. Universal nucleotides comprise universal bases that can bind to any nucleotide. Non-limiting examples of universal bases comprise inosine, hypoxanthine, nitroazoles, isocarbostyril analogues, azole carboxamides, or aromatic triazole analogues (see e.g., Loakes et al., Nucleic Acids Res. 2001 Jun. 15; 29(12):2437-47; Berger et al., Nucleic Acids Res. 2000 Aug. 1; 28(15): 2911-2914; Liang et al., RSC Advances 3(35); June 2013).

In some embodiments of any of the aspects, a readout pool comprises at least 2 sets of readout molecules, wherein each set is linked to the same type of detection molecule, which is distinct from the detection molecule linked to the other set(s) of readout molecules, and each set detects the same nucleotide in the barcode region, which is distinct from the nucleotide in the same position of the barcode region detected by the other set(s) of readout molecules. In some embodiments of any of the aspects, a readout pool comprises at least 1 set, 2 sets, 3 sets, 4 sets, or at least 5 sets of readout molecules.

In some embodiments of any of the aspects, the sample is contacted with a first readout pool that recognizes the first nucleotide of the barcode region of each cassette (or that recognizes different cassettes at a first cassette position in a street). In some embodiments of any of the aspects, the sample is contacted with a second readout pool that recognizes the second nucleotide of the barcode region of each cassette (or that recognizes different cassettes at a second cassette position in a street). In some embodiments of any of the aspects, the sample is contacted with a third readout pool that recognizes the third nucleotide of the barcode region of each cassette (or that recognizes different cassettes at a third cassette position in a street). In some embodiments of any of the aspects, the sample is contacted with a fourth readout pool that recognizes the fourth nucleotide of the barcode region of each cassette (or that recognizes different cassettes at a fourth cassette position in a street). In some embodiments of any of the aspects, the sample is contacted with a fifth readout pool that recognizes the fifth nucleotide of the barcode region of each cassette (or that recognizes different cassettes at a fifth cassette position in a street). In some embodiments of any of the aspects, the sample is contacted with a n^{th} readout pool that recognizes the n^{th} nucleotide of the barcode region of each cassette (or that recognizes different cassettes at a n^{th} cassette position in a street), where n corresponds to an integer from 1 to 10.

In some embodiments of any of the aspects, the sample is contacted with each readout pool sequentially. In some embodiments of any of the aspects, in between contacting the sample with an n^{th} readout pool and an (n+1)^{th} readout pool, the readout pool is detected as described herein, and the n^{th} readout pool is washed away (e.g., with any buffer appropriate for use in hybridization reactions, e.g., 60% formamide in 2× SSCT, wherein SSC refers to saline-sodium citrate buffer and T refers to TWEEN).

In some embodiments of any of the aspects, the sample is contacted with at least two readout pools concurrently. As a non-limiting example, the sample is contacted concurrently with at least 2 readout pools, at least 3 readout pools, at least 4 readout pools, or at least 5 readout pools. Compared to contacting a sample with one readout pool, contacting a sample with at least two readout pools concurrently can provide added benefits including but not limited to amplification of the signal, introduction of additional optically detectable markers (e.g., psuedocolor combinations of different fluorophores), and increased speed of the process.

In some embodiments of any of the aspects, the method of analyzing at least one target molecule in a sample comprises contacting the sample with at least one oligonucleotide tag (e.g., an Oligopaint), contacting the sample with at least two readout molecules, and detecting the relative spatial order of the readout molecules. In some embodiments of any of the aspects, the specific hybridization of a readout molecule to a cassette is determined by or is dependent on the identity of the barcode region.

In some embodiments of any of the aspects, compositions and methods described herein comprise improvements of compositions and methods related to Oligopaint technology. As used herein, the term “Oligopaint” refers to polynucleotides that have sequences complementary to a target molecule, e.g., an oligonucleotide sequence, a portion of a DNA sequence, or a particular chromosome or sub-chromosomal region of a particular chromosome.

Traditionally, fluorescence in situ hybridization (FISH) probes are derived from cloned genomic regions or flow-sorted chromosomes, which are labeled directly via nick translation or PCR in the presence of fluorophore-conjugated nucleotides or labeled indirectly with nucleotide-conjugated haptens, such as biotin and digoxigenin, and then visualized with secondary detection reagents. Traditional FISH probes are limited by repetitive sequences and variable efficacy. Furthermore, target regions are restricted by the availability of clones and the size of their genomic inserts. Whereas it is possible to target larger regions with traditional FISH probes, this approach is often challenging and expensive, as each clone needs to be prepared and optimized for hybridization separately.

Oligopaints are an improved FISH technology wherein oligo libraries can be produced by massively parallel synthesis can be used as a renewable source of probes. Oligo libraries can be PCR-amplified (optionally with fluorophore-conjugated primers). The amplification products can be enzymatically processed to produce highly efficient single-stranded, strand-specific probes that can visualize regions ranging from tens of kilobases to megabases. Oligopaints can comprise synthetic probes and arrays that are, optionally, computationally patterned and/or computationally designed.

For publications directed at Oligopaint and related technologies, see e.g., Beliveau et al. OligoMiner provides a rapid, flexible environment for the design of genome-scale oligonucleotide in situ hybridization probes. Proc. Nat. Acad. Sci. USA 2018 115:E2183-E2192; Beliveau et al. In situ super-resolution imaging of genomic DNA with OligoSTORM and OligoDNA-PAINT. Methods Mol Biol 2017 1663:231-252; Wang et al. Spatial organization of chromatin domains and compartments in single chromosomes. Science 2016 353:598-602; Boettiger et al. Super-resolution imaging reveals distinct chromatin folding for different epigenetic states. Nature. 2016 529:418-22; Schmidt et al. Scalable amplification of strand subsets from chip-synthesized oligonucleotide libraries. Nat Commun 2015 Nov. 16; 6:8634; Murgha et al. Combined in vitro transcription and reverse transcription method to amplify and label complex synthetic oligonucleotide probe libraries. Biotechniques 2015 58:301-7; Beliveau et al. Single-molecule super-resolution imaging of chromosomes and in situ haplotype visualization using Oligopaint FISH probes. Nat Commun 2015 6:7147; Beliveau et al. Visualizing genomes with Oligopaint FISH probes. Curr Protocols Mol Biol 2014 14.23; Beliveau et al. A versatile design and synthesis platform for visualizing genomes with Oligopaint FISH probes. Proc. Nat. Acad. Sci. USA 2012 109:21301-6; US 2010/0304994 A1; US 2018/0223347 A1; WO 2018/045186 A1; US 2014/0364333 A1; US 2019/0032121 A1; US 2013/0143208 A1; U.S. Pat. No. 10,119,160 B2; US 2018/0057867 A1; US 2019/0127786 A1; US 2018/0292318 A1; WO 2017/189525 A1; WO 2018/183851 A1; WO 2018/183860 A1; WO 2018/045181 A1; US 2016/0040235 A1; each of which is incorporated herein by reference in its entirety.

As used herein, the terms “Oligopainted” and “Oligopainted region” refer to a target nucleotide sequence (e.g., a chromosome) or region of a target nucleotide sequence (e.g., a sub-chromosomal region), respectively, that has hybridized with one or more Oligopaints. Oligopaints can be used to label a target nucleotide sequence, e.g., chromosomes and sub-chromosomal regions of chromosomes during various phases of the cell cycle including, but not limited to, interphase, preprophase, prophase, prometaphase, metaphase, anaphase, telophase and cytokinesis.

In some embodiments of any of the aspects, FISH methods can comprise Oligopaint, multiplexed error-robust fluorescence in situ hybridization (MERFISH), seqFISH, RNA sequential probing of targets (SPOTs), high-coverage microscopy-based technology (Hi-M), or optical reconstruction of chromatin architecture (ORCA) or any method comprising contacting a sample with a oligonucleotide that has a sequence complementary (e.g. recognition domain) to a target molecule, e.g., an oligonucleotide sequence, a portion of a DNA sequence, or a particular chromosome or sub-chromosomal region of a particular chromosome. For further details, see e.g., Cardozo et al., Mol Cell. 2019 Apr. 4; 74(1):212-222; Mateo et al., Nature. 2019 April; 568(7750):49-54; Wang et al., Scientific Reports volume 8, Article number: 4847 (2018); Shah et al., Neuron, Volume 92, Issue 2, 19 Oct. 2016, Pages 342-357; Eng et al., Nat Methods. 2017 December; 14(12):1153-1155; each of which is incorporated herein by reference in its entirety.

In some embodiments of any of the aspects, of the methods described herein comprise analyzing at least one target molecule in a sample. In some embodiments of any of the aspects, the target molecule comprises a nucleic acid, a polypeptide, a cell surface molecule, and/or an inorganic material. In some embodiments of any of the aspects, the target molecule comprises DNA, including but not limited to genomic DNA, genomic DNA organized as chromosomes, or complementary DNA (cDNA). In some embodiments of any of the aspects, the target molecule comprises RNA, including but not limited to messenger RNA (mRNA) or ribosomal RNA (rRNA). In some embodiments of any of the aspects, the target molecule comprises a polypeptide, including but not limited to intracellular proteins, transmembrane proteins, or extracellular proteins. In some embodiments of any of the aspects, the target molecule comprises a cell surface molecule, including but not limited to transmembrane proteins, membrane lipids, membrane receptors, or transmembrane receptors. In some embodiments of any of the aspects, the target molecule comprises an inorganic material comprising any material derived from a non-living source, including but not limited to glass, ceramics, metals, or any other solid substrate.

In some embodiments of any of the aspects, one target molecule is analyzed. In some embodiments of any of the aspects, at least two target molecules are analyzed concurrently. As a non-limiting example, at least 2 target molecules, at least 3 target molecules, at least 4 target molecules, at least 5 target molecules, at least 6 target molecules, at least 7 target molecules, at least 8 target molecules, at least 9 target molecules, at least 10 target molecules, or at least 20 target molecules are analyzed concurrently.

In some embodiments of any of the aspects, more than one region of a target molecule is analyzed concurrently. As a non-limiting example, 2 regions, 3 regions, 4 regions, 5 regions, 6 regions, 7 regions, 8 regions, 9 regions, 10 regions, or greater than 10 regions of a target molecule or target molecules are analyzed.

In some embodiments of any of the aspects, the sample is a cell, cell culture, or tissue sample. As a non-limiting example the cell, cell culture, or tissue sample is taken at a time or under conditions in which individual chromosomes are distinguishable, e.g., mitosis.

The term “sample” or “test sample” as used herein denotes a sample taken or isolated from a biological organism, e.g., a blood or tissue sample from a subject. In some embodiments of any of the aspects, the present invention encompasses several examples of a biological sample. In some embodiments of any of the aspects, the biological sample is cells, or tissue, or peripheral blood, or bodily fluid. Exemplary biological samples include, but are not limited to, a biopsy, a tumor sample, biofluid sample; blood; serum; plasma; urine; sperm; mucus; tissue biopsy; organ biopsy; synovial fluid; bile fluid; cerebrospinal fluid; mucosal secretion; effusion; sweat; saliva; and/or tissue sample etc. The term also includes a mixture of the above-mentioned samples. The term “test sample” also includes untreated or pretreated (or pre-processed) biological samples. In some embodiments of any of the aspects, a test sample comprises cells from a subject.

The test sample can be obtained by removing a sample from a subject, but can also be accomplished by using a previously isolated sample (e.g. isolated at a prior time point and isolated by the same or another person).

In some embodiments of any of the aspects, the test sample can be an untreated test sample. As used herein, the phrase “untreated test sample” refers to a test sample that has not had any prior sample pre-treatment except for dilution and/or suspension in a solution. Exemplary methods for treating a test sample include, but are not limited to, centrifugation, filtration, sonication, homogenization, heating, freezing and thawing, and combinations thereof. In some embodiments of any of the aspects, the test sample can be a frozen test sample, e.g., a frozen tissue. The frozen sample can be thawed before employing methods, assays and systems described herein. After thawing, a frozen sample can be centrifuged before being subjected to methods, assays and systems described herein. In some embodiments of any of the aspects, the test sample is a clarified test sample, for example, by centrifugation and collection of a supernatant comprising the clarified test sample. In some embodiments of any of the aspects, a test sample can be a pre-processed test sample, for example, supernatant or filtrate resulting from a treatment selected from the group consisting of centrifugation, filtration, thawing, purification, and any combinations thereof. In some embodiments of any of the aspects, the test sample can be treated with a chemical and/or biological reagent. Chemical and/or biological reagents can be employed to protect and/or maintain the stability of the sample, including biomolecules (e.g., nucleic acid and protein) therein, during processing. One exemplary reagent is a protease inhibitor, which is generally used to protect or maintain the stability of protein during processing. The skilled artisan is well aware of methods and processes appropriate for pre-processing of biological samples required for determination of the level of an expression product as described herein.

In some embodiments of any of the aspects, the methods, assays, and systems described herein can further comprise a step of obtaining or having obtained a test sample from a subject. In some embodiments of any of the aspects, the subject can be a human subject.

In some embodiments of any of the aspects, the oligonucleotide tag (e.g., an Oligopaint) comprises a recognition domain. As used herein, a “recognition domain” is a domain of the oligonucleotide tag (e.g., an Oligopaint) that binds specifically to a target molecule and/or sequence to be analyzed. As a non-limiting example, the recognition domain can be a nucleic acid sequence that is complementary to a target molecule and/or sequence, e.g., a region of a chromosome. Accordingly, the sequence of the recognition domain will vary depending on the identity of the desired target. It is well within the skill of the art to design a recognition domain that will specifically hybridize to any given target under specific conditions, e.g., using software widely and freely available for this purpose (e.g., Primer3 or PrimerBank, which are both available on the world wide web). In some embodiments of any of the aspects, the recognition domain can have at least 90%, at least 91%, at least 92%, at least 93%, at least 94%, at least 95%, at least 96%, at least 97%, at least 98%, at least 99%, or 100% identity with a portion of the target molecule and/or with the target sequence. In some embodiments of any of the aspects, the recognition domain comprises a domain of “genomic homology” or a domain that specifically binds to a region of the genome. In some embodiments of any of the aspects, multiple recognition domains found on the same or different oligonucleotide tags (e.g., an Oligopaint) can specifically bind to a single target molecule and/or target sequence. As a non-limiting example, at least 2 recognition domains, at least 3 recognition domains, at least 4 recognition domains, at least 5 recognition domains, at least 10 recognition domains, at least 20 recognition domains, at least 30 recognition domains, at least 40 recognition domains, or at least 50 recognition domains can specifically bind to a target molecule and/or target sequence.

In some embodiments of any of the aspects, the recognition domain comprises or is comprised by oligonucleotides including but not limited to Oligopaints, multiplexed error-robust fluorescence in situ hybridization (MERFISH) oligos, seqFISH oligos, RNA sequential probing of targets (SPOTs) oligos, high-coverage microscopy-based technology (Hi-M) oligos, or optical reconstruction of chromatin architecture (ORCA) oligos or any to oligonucleotide used for FISH methods and/or any oligonucleotides that has a sequence complementary (e.g. recognition domain) to a target molecule, e.g., an oligonucleotide sequence, a portion of a DNA sequence, or a particular chromosome or sub-chromosomal region of a particular chromosome. For further details, see e.g., Cardozo et al., Mol Cell. 2019 Apr. 4; 74(1):212-222; Mateo et al., Nature. 2019 April; 568(7750):49-54; Wang et al., Scientific Reports volume 8, Article number: 4847 (2018); Shah et al., Neuron, Volume 92, Issue 2, 19 Oct. 2016, Pages 342-357; Eng et al., Nat Methods. 2017 December; 14(12):1153-1155; each of which is incorporated herein by reference in its entirety.

In some embodiments of any of the aspects, the recognition domain comprises a non-nucleic acid, e.g., any nucleic-acid binding composition such as a DNA-binding polypeptide. As a non-limiting example, the recognition domain comprises a sequence-specific single-stranded DNA binding protein or factor, a sequence-specific double-stranded DNA binding protein or factor, a DNA-RNA binding protein or factor, or an RNA binding protein or factor. Non-limiting examples of such a nucleic-acid-binding composition include but are not limited to a transcription factor, a restriction enzyme, a transcription activator-like effector nuclease (TALENs), a CRISPR-Cas-type factor, and the like. In some embodiments of any of the aspects, the nucleic-acid-binding composition lacks nuclease activity.

In some embodiments of any of the aspects, the target molecule comprises a non-nucleic acid, e.g., a polypeptide. Accordingly, the recognition domain comprises any composition that specifically binds a target polypeptide. Non-limiting examples of such a polypeptide-binding recognition domain include but are not limited to an antibody (e.g., a nanobody), an aptamer, a small molecule, a ligand, a known binding partner of a specific polypeptide, and the like.

In some embodiments of any of the aspects, the oligonucleotide tag does not specifically recognize a target molecule, in that the oligonucleotide tag is not linked to a recognition domain but is linked to an entity for detecting the oligonucleotide-tagged entity of interest. The oligonucleotide tag can be a nucleic acid comprising at one anchor region and at least barcode region, but e.g., lacking a recognition domain. As non-limiting examples, such oligonucleotide-tagged entities can include small molecules (e.g., for the purpose of drug screens), polypeptides, cells, or non-biological materials (e.g., metals, chemicals, etc.). The methods of detecting such oligonucleotide-tagged entities can be identical to those used for detecting oligonucleotide tags (e.g., Oligopaint) as described herein (e.g., contacting with pools of readout molecules that hybridize to the specific cassette types in the oligonucleotide tags). In some embodiments of any of the aspects, multiple types of target molecules and/or types of tagged entities can be detected at once, e.g., using at least one oligonucleotide tag (e.g., Oligopaint) that recognizes DNA, at least one oligonucleotide tag that recognizes polypeptides, and/or at least one- oligonucleotide-tagged entity.

In some embodiments of any of the aspects, the oligonucleotide tag (e.g., Oligopaint) comprises at least one street. As used herein, the term “street” refers to a portion of the oligonucleotide tag (e.g., Oligopaint) that does not have identity with a target sequence or does not hybridize to a target sequence. Streets comprises regions for detection and/or regions for amplification. As a non-limiting example, the oligonucleotide tag (e.g., Oligopaint) comprises two streets. In some embodiments of any of the aspects, the street can be one or more of a “Mainstreet” and/or a “Backstreet”. As a non-limiting example, the Mainstreet is 5′ to the recognition domain, the Mainstreet is 5′ to the Backstreet, and/or the Mainstreet is 5′ to the recognition domain and the Backstreet. As a non-limiting example, the Backstreet is 3′ to the recognition domain, the Backstreet is 3′ to the Mainstreet, and/or the Backstreet is 3′ to the recognition domain and the Mainstreet. As a non-limiting example, the Mainstreet is 3′ to the recognition domain, the Mainstreet is 3′ to the Backstreet, and/or the Mainstreet is 3′ to the recognition domain and the Backstreet. As a non-limiting example, the Backstreet is 5′ to the recognition domain, the Backstreet is 5′ to the Mainstreet, and/or the Backstreet is 5′ to the recognition domain and the Mainstreet.

In some embodiments of any of the aspects, the street (e.g., Mainstreet and/or Backstreet) comprises at least one cassette and/or at least one universal priming region. As described herein, said cassette comprises at least one barcode region and at least one anchor region. As used herein, “universal priming region” refers to a region that binds a universal primer (e.g., a universal forward primer, a universal reverse primer). As used herein, “universal primer” refers to a primer that is used for multiple individual oligonucleotide tags (e.g., Oligopaint) or a set of oligonucleotide tags. Universal primers can be used for the purpose of amplifying, for example with PCR, the oligonucleotide tag (e.g., Oligopaint), e.g., for production of the oligonucleotide tag or set of oligonucleotide tags. In some embodiments of any of the aspects, the universal priming region of each oligonucleotide tag (e.g., Oligopaint) is identical to the universal priming region of the remaining oligonucleotide tags, e.g., any other oligonucleotide tag the sample is contacted with.

In some embodiments of any of the aspects, the street comprises at least one universal priming region and/or at least one cassette. As a non-limiting example, the universal priming region is 5′ of at least one cassette. As a non-limiting example, the universal forward priming region, which specifically binds to a universal forward primer, is at the 5′ end of the oligonucleotide tag (e.g., Oligopaint). As a non-limiting example, the universal priming region is 3′ of at least one cassette. As a non-limiting example, the universal reverse priming region, which specifically binds to a universal reverse primer, is at the 3′ end of the oligonucleotide tag (e.g., Oligopaint). In some embodiments of any of the aspects, universal priming regions flank (both 5′ and 3′) any cassettes present in the oligonucleotide tag (e.g., Oligopaint). In some embodiments of any of the aspects, the universal reverse priming region comprises a recognition site for a nicking endonuclease (NE), e.g., to cause the oligonucleotide tag (e.g., Oligopaint) to become single-stranded when exposed to an NE. In some embodiments of any of the aspects, the oligonucleotide tag (e.g., Oligopaint) is not necessarily amplified (e.g., through PCR and/or universal priming regions). In some embodiments of any of the aspects, the oligonucleotide tag (e.g., Oligopaint) described can be synthesized, de novo, and used “straight from the tube”.

In some embodiments of any of the aspects, the detecting is performed with at least single cell resolution. As a non-limiting example, the detecting can be performed with a resolution of at least 200 nm, at least 300 nm, at least 400 nm, at least 500 nm, at least 600 nm, at least 700 nm, at least 800 nm, at least 900 nm, at least 1 μm, at least 2 μm, at least 3 μm, at least 4 μm, at least 5 μm, at least 6 μm, at least 7 μm, at least 8 μm, at least 9 μm, or at least 10 μm. In some embodiments of any of the aspects, the detecting is performed with a resolution that can differentiate individual target molecules, e.g., chromosomes.

In some embodiments of any of the aspects, the nucleic acid e.g., an oligonucleotide tag (e.g., Oligopaint), is chemically modified to enhance stability or other beneficial characteristics. The nucleic acids described herein may be synthesized and/or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference. Modifications include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position) or replacement of the sugar, as well as (d) backbone modifications, including modification or replacement of the phosphodiester linkages. Specific examples of nucleic acid compounds useful in the embodiments described herein include, but are not limited to nucleic acids containing modified backbones or no natural internucleoside linkages. nucleic acids having modified backbones include, among others, those that do not have a phosphorus atom in the backbone. For the purposes of this specification, and as sometimes referenced in the art, modified nucleic acids that do not have a phosphorus atom in their internucleoside backbone can also be considered to be oligonucleosides. In some embodiments of any of the aspects, the modified nucleic acid will have a phosphorus atom in its internucleoside backbone.

Modified nucleic acid backbones can include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included. Modified nucleic acid backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; others having mixed N, O, S and CH2 component parts, and oligonucleosides with heteroatom backbones, and in particular —CH2-NH—CH2-, —CH2-N(CH3)-O—CH2-[known as a methylene (methylimino) or MMI backbone], —CH2-O—N(CH3)-CH2-, —CH2-N(CH3)-N(CH3)-CH2- and —N(CH3)-CH2-CH2-[wherein the native phosphodiester backbone is represented as —O—P—O—CH2-].

Modified nucleic acids can also contain one or more substituted sugar moieties. The nucleic acids described herein can include one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. Exemplary suitable modifications include O[(CH2)nO] mCH3, O(CH2).nOCH3, O(CH2)nNH2, O(CH2) nCH3, O(CH2)nONH2, and O(CH2)nON[(CH2)nCH3)]2, where n and m are from 1 to about 10. In some embodiments of any of the aspects, dsRNAs include one of the following at the 2′ position: C1 to C10 lower alkyl, substituted lower alkyl, alkaryl, aralkyl, O-alkaryl or O-aralkyl, SH, SCH3, OCN, Cl, Br, CN, CF3, OCF3, SOCH3, SO2CH3, ONO2, NO2, N3, NH2, heterocycloalkyl, heterocycloalkaryl, aminoalkylamino, polyalkylamino, substituted silyl, an RNA cleaving group, a reporter group, an intercalator, a group for improving the pharmacokinetic properties a nucleic acid, or a group for improving the pharmacodynamic properties of a nucleic acid, and other sub stituents having similar properties. In some embodiments of any of the aspects, the modification includes a 2′ methoxyethoxy (2′-O-CH2CH2OCH3, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH2)2ON(CH3)2 group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O-CH2-O—CH2-N(CH2)2, also described in examples herein below.

Other modifications include 2′-methoxy (2′-OCH3), 2′-aminopropoxy (2′-OCH2CH2CH2NH2) and 2′-fluoro (2′-F). Similar modifications can also be made at other positions on the nucleic acid, particularly the 3′ position of the sugar on the 3′ terminal nucleotide or in 2′-5′ linked dsRNAs and the 5′ position of 5′ terminal nucleotide. Nucleic acids may also have sugar mimetics such as cyclobutyl moieties in place of the pentofuranosyl sugar.

A nucleic acid can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases can include ther synthetic and natural nucleobases including but not limited to 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Certain of these nucleobases are particularly useful for increasing the binding affinity of the inhibitory nucleic acids featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications. In some embodiments of any of the aspects, modified nucleobases can include d5SICS and dNAM, which are a non-limiting example of unnatural nucleobases that can be used separately or together as base pairs (see e.g., Leconte et. al. J. Am. Chem. Soc. 2008, 130, 7, 2336-2343; Malyshev et. al. PNAS. 2012. 109 (30) 12005-12010). In some embodiments of any of the aspects, oligonucleotide tags (e.g., Oligopaint) comprise any modified nucleobases known in the art, i.e., any nucleobase that is modified from an unmodified and/or natural nucleobase.

The preparation of the modified nucleic acids, backbones, and nucleobases described above are well known in the art.

Another modification of a nucleic acid featured in the invention involves chemically linking to the nucleic acid to one or more ligands, moieties or conjugates that enhance the activity, cellular distribution, pharmacokinetic properties, or cellular uptake of the nucleic acid. Such moieties include but are not limited to lipid moieties such as a cholesterol moiety (Letsinger et al., Proc. Natl. Acid. Sci. USA, 1989, 86: 6553-6556), cholic acid (Manoharan et al., Biorg. Med. Chem. Let., 1994, 4:1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

In some embodiments of any of the aspects, measurement, and/or detection of a target molecule, e.g. a DNA target molecule, an RNA target molecule, or a polypeptide target molecule comprises contacting a sample obtained from a subject with a reagent or reagents as described herein. In some embodiments of any of the aspects, the reagent is detectably labeled. In some embodiments of any of the aspects, the reagent is capable of generating a detectable signal. In some embodiments of any of the aspects, the reagent generates a detectable signal when the target molecule is present.

In some embodiments of any of the aspects, one or more of the reagents described herein can comprise a detectable label and/or comprise the ability to generate a detectable signal (e.g. by catalyzing reaction converting a compound to a detectable product). Detectable labels can comprise, for example, a light-absorbing dye, a fluorescent dye, or a radioactive label. Detectable labels, methods of detecting them, and methods of incorporating them into reagents described herein are well known in the art.

In some embodiments of any of the aspects, detectable labels, molecules, and/or moieties can include those that can be detected by spectroscopic, photochemical, biochemical, immunochemical, electromagnetic, radiochemical, or chemical means, such as fluorescence, chemifluorescence, or chemiluminescence, or any other appropriate means. The detectable labels used in the methods described herein can be primary labels (where the label comprises a moiety that is directly detectable or that produces a directly detectable moiety) or secondary labels (where the detectable label binds to another moiety to produce a detectable signal, e.g., as is common in immunological labeling using secondary and tertiary antibodies). The detectable label can be linked by covalent or non-covalent means to the reagent. Alternatively, a detectable label can be linked such as by directly labeling a molecule that achieves binding to the reagent via a ligand-receptor binding pair arrangement or other such specific recognition molecules. Detectable labels can include, but are not limited to radioisotopes, bioluminescent compounds, chromophores, antibodies, chemiluminescent compounds, fluorescent compounds, metal chelates, and enzymes.

In other embodiments, the detection reagent is label with a fluorescent compound. When the fluorescently labeled reagent is exposed to light of the proper wavelength, its presence can then be detected due to fluorescence. In some embodiments of any of the aspects, a detectable label can be a fluorescent dye molecule, or fluorophore including, but not limited to fluorescein, phycoerythrin, phycocyanin, o-phthalaldehyde, fluorescamine, Cy3™, Cy5™, allophycocyanin, Texas Red, peridinin chlorophyll, cyanine, tandem conjugates such as phycoerythrin-Cy5™, green fluorescent protein, rhodamine, fluorescein isothiocyanate (FITC) and Oregon Green™, rhodamine and derivatives (e.g., Texas red and tetrarhodimine isothiocyanate (TRITC)), biotin, phycoerythrin, AMCA, CyDyes™, 6-carboxyfhiorescein (commonly known by the abbreviations FAM and F), 6-carboxy-2′,4′,7′,4,7-hexachlorofiuorescein (HEX), 6-carboxy-4′,5′-dichloro-2′,7′-dimethoxyfiuorescein (JOE or J), N,N,N′,N′-tetramethyl-6carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), and rhodamine 110; cyanine dyes, e.g. Cy3, Cy5 and Cy7 dyes; coumarins, e.g., umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3, Cy5, etc.; BODIPY dyes and quinoline dyes. In some embodiments of any of the aspects, a detectable label can be a radiolabel including, but not limited to ³H, ¹²⁵I, ³⁵S, ¹⁴C, ³²P, and ³³P. In some embodiments of any of the aspects, a detectable label can be an enzyme including, but not limited to horseradish peroxidase and alkaline phosphatase. An enzymatic label can produce, for example, a chemiluminescent signal, a color signal, or a fluorescent signal. Enzymes contemplated for use to detectably label an antibody reagent include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase. In some embodiments of any of the aspects, a detectable label is a chemiluminescent label, including, but not limited to lucigenin, luminol, luciferin, isoluminol, theromatic acridinium ester, imidazole, acridinium salt and oxalate ester. In some embodiments of any of the aspects, a detectable label can be a spectral colorimetric label including, but not limited to colloidal gold or colored glass or plastic (e.g., polystyrene, polypropylene, and latex) beads.

In some embodiments of any of the aspects, detection reagents can also be labeled with a detectable tag, such as c-Myc, HA, VSV-G, HSV, FLAG, V5, HIS, or biotin. Other detection systems can also be used, for example, a biotin-streptavidin system. In this system, the antibodies immunoreactive (i. e. specific for) with the biomarker of interest is biotinylated. Quantity of biotinylated antibody bound to the biomarker is determined using a streptavidin-peroxidase conjugate and a chromogenic substrate. Such streptavidin peroxidase detection kits are commercially available, e. g. from DAKO; Carpinteria, Calif. A reagent can also be detectably labeled using fluorescence emitting metals such as ¹⁵²Eu, or others of the lanthanide series. These metals can be attached to the reagent using such metal chelating groups as diethylenetriaminepentaacetic acid (DTPA) or ethylenediaminetetraacetic acid (EDTA).

Detection method(s) used will depend on the particular detectable labels used in the readout molecules. In certain exemplary embodiments, chromosomes and/or chromosomal regions having one or more oligonucleotide tags (e.g., Oligopaint) and/or readout molecules bound thereto may be selected for and/or screened for using a microscope, a spectrophotometer, a tube luminometer or plate luminometer, x-ray film, a scintillator, a fluorescence activated cell sorting (FACS) apparatus, a microfluidics apparatus or the like.

In some embodiments of any of the aspects, the detection molecules comprise fluorophores or fluorescent compounds. Systems and devices for the measurement of fluorescence are well known in the art. Fluorescence measurement requires a light source that emits light comprising the appropriate absorption or excitation wavelength. The absorption or excitation wavelength of the compounds described herein is approximately 300-800 nm. In some embodiments of any of the aspects, the light source emits light comprising, consisting essentially of, or consisting of a wavelength of 300-870 nm. The light contacts the sample, which excites electrons in certain materials within the sample, also known as fluorophores, and causes the materials to emit light (light emission) in the form of fluorescence.

The system or device for measurement of fluorescence then detects the emitted light. In some embodiments, the system or device can comprise a filter or monochromator so that only light of desired wavelengths reaches the detector of the system or device. In some embodiments of any of the aspects, the system or device is configured to detect light comprising, consisting essentially of, or consisting of a wavelength of 300-800 nm. In some embodiments of any of the aspects, the system or device is configured to detect light comprising, consisting essentially of, or consisting of a wavelength of 300-800 nm. Suitable systems and devices are commercially available and can include, e.g., the 20/30 PV™ Microspectrometer or 508 PV™ Microscope Spectrometer from CRAIC (San Dimas, Calif.), the Duetta™, FluoroMax™, Fluorolog™, QuantaMaster 8000™, DeltaFlex™, DeltaPro, or Nanolog™ from Horiba (Irvine, Calif.), or the SP8 Lightning™, SP8 Falcon™, SP8 Dive™, TCS SPE™, HCS A™, or TCS SP8 X™ from Leica (Buffalo Grove, Ill.).

In some embodiments of any of the aspects, fluorescence photomicroscopy can be used to detect and record the results of in situ hybridization using routine methods known in the art. Alternatively, digital (computer implemented) fluorescence microscopy with image-processing capability may be used. Two well-known systems for imaging FISH of chromosomes having multiple colored labels bound thereto include multiplex-FISH (M-FISH) and spectral karyotyping (SKY). See Schrock et al. (1996) Science 273:494; Roberts et al. (1999) Genes Chrom. Cancer 25:241; Fransz et al. (2002) Proc. Natl. Acad. Sci. USA 99:14584; Bayani et al. (2004) Curr. Protocol. Cell Biol. 22.5.1-22.5.25; Danilova et al. (2008) Chromosoma 117:345; U.S. Pat. No. 6,066,459; and FISH TAG™ DNA Multicolor Kit instructions (Molecular probes) for a review of methods for painting chromosomes and detecting painted chromosomes.

In certain exemplary embodiments, images of fluorescently labeled chromosomes are detected and recorded using a computerized imaging system such as the Applied Imaging Corporation CytoVision™ System (Applied Imaging Corporation, Santa Clara, Calif.) with modifications (e.g., software, Chroma 84000 filter set, and an enhanced filter wheel). Other suitable systems include a computerized imaging system using a cooled CCD camera (Photometrics, NU200 series equipped with Kodak KAF 1400 CCD) coupled to a Zeiss Axiophot microscope, with images processed as described by Ried et al. (1992) Proc. Natl. Acad. Sci. USA 89:1388). Other suitable imaging and analysis systems are described by Schrock et al., supra; and Speicher et al. (1996) Nature Genet. 12:368. In some embodiments of any of the aspects, the oligonucleotide tags (e.g., Oligopaint) are visualized with super resolution microscopy (e.g. Stochastic Optical Reconstruction Microscopy (STORM) Imaging).

The in situ hybridization methods described herein can be performed on a variety of biological or clinical samples, in cells that are in any (or all) stage(s) of the cell cycle (e.g., mitosis, meiosis, interphase, G0, G1, S and/or G2). Examples include all types of cell culture, animal or plant tissue, peripheral blood lymphocytes, buccal smears, touch preparations prepared from uncultured primary tumors, cancer cells, bone marrow, cells obtained from biopsy or cells in bodily fluids (e.g., blood, urine, sputum and the like), cells from amniotic fluid, cells from maternal blood (e.g., fetal cells), cells from testis and ovary, and the like. Samples are prepared for assays of the invention using conventional techniques, which typically depend on the source from which a sample or specimen is taken. These examples are not to be construed as limiting the sample types applicable to the methods and/or compositions described herein.

Hybridization of the oligonucleotide tags (e.g., Oligopaint) of the invention to target chromosomes sequences can be accomplished by standard in situ hybridization (ISH) techniques (see, e.g., Gall and Pardue (1981) Meth. Enzymol. 21:470; Henderson (1982) Int. Review of Cytology 76:1). Generally, ISH comprises the following major steps: (1) fixation of the biological structure to be analyzed (e.g., a chromosome spread), (2) pre-hybridization treatment of the biological structure to increase accessibility of target DNA (e.g., denaturation with heat or alkali), (3) optional pre-hybridization treatment to reduce nonspecific binding (e.g., by blocking the hybridization capacity of repetitive sequences), (4) hybridization of the mixture of nucleic acids to the nucleic acid in the biological structure or tissue; (5) post-hybridization washes to remove nucleic acid fragments not bound in the hybridization and (6) detection of the hybridized labeled oligonucleotides (e.g., hybridized oligonucleotide tags, e.g., Oligopaints). The reagents used in each of these steps and their conditions of use vary depending on the particular situation. For instance, step 3 will not always be necessary as the recognition domains described herein can be designed to avoid repetitive sequences). Hybridization conditions are also described in U.S. Pat. No. 5,447,841. It will be appreciated that numerous variations of in situ hybridization protocols and conditions are known and may be used in conjunction with the present invention by practitioners following the guidance provided herein.

As used herein, the term “hybridization” refers to the process in which two single-stranded polynucleotides bind non-covalently to form a stable double-stranded polynucleotide. The term “hybridization” may also refer to triple-stranded hybridization. The resulting (usually) double-stranded polynucleotide is a “hybrid” or “duplex.” “Hybridization conditions” will typically include salt concentrations of less than about 1 M, more usually less than about 500 mM and even more usually less than about 200 mM. Hybridization temperatures can be as low as 5° C., but are typically greater than 22° C., more typically greater than about 30° C., and often in excess of about 37° C. Hybridizations are usually performed under stringent conditions, i.e., conditions under which a probe will hybridize to its target subsequence. Stringent conditions are sequence-dependent and are different in different circumstances. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone. Generally, stringent conditions are selected to be about 5° C. lower than the Tm for the specific sequence at s defined ionic strength and pH. Exemplary stringent conditions include salt concentration of at least 0.01 M to no more than 1 M Na ion concentration (or other salts) at a pH 7.0 to 8.3 and a temperature of at least 25° C. For example, conditions of 5×SSPE (750 mM NaCl, 50 mM Na phosphate, 5 mM EDTA, pH 7.4) and a temperature of 25-30° C. are suitable for allele-specific probe hybridizations. For stringent conditions, see for example, Sambrook, Fritsche and Maniatis, Molecular Cloning A Laboratory Manual, 2nd Ed. Cold Spring Harbor Press (1989) and Anderson Nucleic Acid Hybridization, 1st Ed., BIOS Scientific Publishers Limited (1999). “Hybridizing specifically to” or “specifically hybridizing to” or like expressions refer to the binding, duplexing, or hybridizing of a molecule substantially to or only to a particular nucleotide sequence or sequences under stringent conditions when that sequence is present in a complex mixture (e.g., total cellular) DNA or RNA.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third nontarget entity. A reagent specific for a given target is one that exhibits specific binding for that target under the conditions of the assay being utilized.

As used herein, the term “oligonucleotide” is intended to include, but is not limited to, a single-stranded DNA or RNA molecule, typically prepared by synthetic means. Nucleotides of the present invention will typically be the naturally-occurring nucleotides such as nucleotides derived from adenosine, guanosine, uridine, cytidine and thymidine. When oligonucleotides are referred to as “double-stranded,” it is understood by those of skill in the art that a pair of oligonucleotides exists in a hydrogen-bonded, helical array typically associated with, for example, DNA. In addition to the 100% complementary form of double-stranded oligonucleotides, the term “double-stranded” as used herein is also meant to include those form which include such structural features as bulges and loops (see Stryer, Biochemistry, Third Ed. (1988), incorporated herein by reference in its entirety for all purposes). As used herein, the term “polynucleotide” is intended to include, but is not limited to, two or more oligonucleotides joined together (e.g., by hybridization, ligation, polymerization and the like).

Nucleic acid and ribonucleic acid (RNA) molecules can be isolated from a particular biological sample using any of a number of procedures, which are well-known in the art, the particular isolation procedure chosen being appropriate for the particular biological sample. For example, freeze-thaw and alkaline lysis procedures can be useful for obtaining nucleic acid molecules from solid materials; heat and alkaline lysis procedures can be useful for obtaining nucleic acid molecules from urine; and proteinase K extraction can be used to obtain nucleic acid from blood (Roiff, A et al. PCR: Clinical Diagnostics and Research, Springer (1994)).

In certain exemplary embodiments, universal primers can be used to amplify nucleic acid sequences such as, for example, oligonucleotide tags (e.g., Oligopaint). The term “universal primers” refers to a set of primers (e.g., a forward and reverse primer) that may be used for chain extension/amplification of a plurality of polynucleotides, e.g., the primers hybridize to sites that are common to a plurality of polynucleotides. For example, universal primers may be used for amplification of all, or essentially all, polynucleotides in a single pool. In some embodiments of any of the aspects, forward primers and reverse primers have the same sequence. In some embodiments of any of the aspects, the sequence of forward primers differs from the sequence of reverse primers. In still other aspects, a plurality of universal primers are provided, e.g., tens, hundreds, thousands or more.

In some embodiments of any of the aspects, the universal primers may be temporary primers that may be removed after amplification via enzymatic or chemical cleavage. In some embodiments of any of the aspects, the universal primers may be temporary primers that may be removed after amplification via enzymatic or chemical cleavage. In other embodiments, the universal primers may comprise a modification that becomes incorporated into the polynucleotide molecules upon chain extension. Exemplary modifications include, for example, a 3′ or 5′ end cap, a label (e.g., fluorescein), or a tag (e.g., a tag that facilitates immobilization or isolation of the polynucleotide, such as, biotin, etc.).

In some embodiments of any of the aspects, the methods disclosed herein comprise amplification of oligonucleotide sequences including, for example, oligonucleotide tags (e.g., Oligopaint). Amplification methods may comprise contacting a nucleic acid with one or more primers (e.g., universal primers) that specifically hybridize to the nucleic acid under conditions that facilitate hybridization and chain extension. Exemplary methods for amplifying nucleic acids include the polymerase chain reaction (PCR) (see, e.g., Mullis et al. (1986) Cold Spring Harb. Symp. Quant. Biol. 51 Pt 1:263 and Cleary et al. (2004) Nature Methods 1:241; and U.S. Pat. Nos. 4,683,195 and 4,683,202), anchor PCR, RACE PCR, ligation chain reaction (LCR) (see, e.g., Landegran et al. (1988) Science 241:1077-1080; and Nakazawa et al. (1994) Proc. Natl. Acad. Sci. U.S.A. 91:360-364), self sustained sequence replication (Guatelli et al. (1990) Proc. Natl. Acad. Sci. U.S.A. 87:1874), transcriptional amplification system (Kwoh et al. (1989) Proc. Natl. Acad. Sci. U.S.A. 86:1173), Q-Beta Replicase (Lizardi et al. (1988) BioTechnology 6:1197), recursive PCR (Jaffe et al. (2000) J. Biol. Chem. 275:2619; and Williams et al. (2002) J. Biol. Chem. 277:7790), the amplification methods described in U.S. Pat. Nos. 6,391,544, 6,365,375, 6,294,323, 6,261,797, 6,124,090 and 5,612,199, or any other nucleic acid amplification method using techniques well known to those of skill in the art. In exemplary embodiments, the methods disclosed herein utilize PCR amplification.

In general, the PCR procedure describes a method of gene amplification which is comprised of (i) sequence-specific hybridization of primers to specific genes or sequences within a nucleic acid sample or library, (ii) subsequent amplification involving multiple rounds of annealing, elongation, and denaturation using a thermostable DNA polymerase, and (iii) screening the PCR products for a band of the correct size. The primers used are oligonucleotides of sufficient length and appropriate sequence to provide initiation of polymerization, i.e. each primer is specifically designed to be complementary to a strand of the genomic locus to be amplified. In an alternative embodiment, mRNA level of gene expression products described herein can be determined by reverse-transcription (RT) PCR and by quantitative RT-PCR (QRT-PCR) or real-time PCR methods. Methods of RT-PCR and QRT-PCR are well known in the art.

In some embodiments of any of the aspects, the oligonucleotide tags (e.g., an Oligopaint) are not necessarily amplified (e.g., through PCR and/or universal priming regions). In some embodiments of any of the aspects, the oligonucleotide tags (e.g., an Oligopaint) described can be synthesized, de novo, and used “straight from the tube”. Methods of synthesizing oligonucleotides de novo are well known to those of skill in the art. As used herein, “oligonucleotide synthesis” refers to the chemical synthesis of relatively short fragments of nucleic acids with defined chemical structure. As a non-limiting example, methods of oligonucleotide synthesis include phosphoramidite solid-phase synthesis, phosphoramidite synthesis, phosphodiester synthesis, phosphotriester synthesis, or phosphite triester synthesis. See e.g., Beaucage et al. Tetrahedron Volume 48, Issue 12, 20 Mar. 1992, Pages 2223-2311; Caruthers, J Biol Chem. 2013 Jan. 11, 288(2):1420-7. In some embodiments, each oligonucleotide is synthesized separately. In some embodiments, the entire oligonucleotide set is synthesized in one reaction. In some embodiments, a subset of the entire oligonucleotide set is synthesized in one reaction. In some embodiments, the entire oligonucleotide set is synthesized in multiple, separate reactions. In some embodiments, reaction products are isolated, e.g., by high-performance liquid chromatography (HPLC), to obtain the desired oligonucleotides in high purity.

In certain exemplary embodiments, kits are provided. As used herein, the term “kit” refers to any delivery system for delivering oligonucleotide tags (e.g., an Oligopaint), readout molecules, and/or reagents for carrying out a method described herein. In the context of assays, such kits include systems that allow for the storage, transport, or delivery of reaction reagents (e.g., an enclosure providing one or more of, e.g., oligonucleotide tags, readout molecules, primers (e.g., primers specific for all oligonucleotide tags present and/or one or more subsets of primers specific to one or more subsets of oligonucleotide tag sequences) primers having one or more detectable and/or retrievable labels bound thereto), supports having oligonucleotides bound thereto (e.g., microarrays, palettes, etc.), or the like) and/or supporting materials (e.g., an enclosure providing, e.g., buffers, written instructions for performing an assay described herein, or the like) from one location to another. For example, kits include one or more enclosures (e.g., boxes) containing the relevant reaction reagents and/or supporting materials for assays described herein. In one aspect, kits of the invention comprise oligonucleotide tags (e.g., an Oligopaint) specific for one or more target nucleotide sequences (e.g., chromosomes) or one or more regions of one or more target nucleotide sequences (e.g., sub-chromosomal regions). In one aspect, kits of the invention comprise readout molecules specific for one or more oligonucleotide tags (e.g., an Oligopaint). In another aspect, kits comprise one or more primer sequences, one or more supports having a plurality of synthetic, oligonucleotide sequences attached thereto, and one or more detectable and/or retrievable labels. Such contents may be delivered to the intended recipient together or separately. For example, a first container may contain primer sequences for use in an assay, while a second container may contain a support having a plurality of synthetic, oligonucleotide sequences attached thereto.

In some embodiments of any of the aspects, a kit provides one or more arrays and/or palettes having a plurality of specific oligonucleotide sequences (e.g., oligonucleotide tags (e.g., an Oligopaint) and/or readout molecules) bound thereto. In some embodiments of any of the aspects, an array and/or palette provides a plurality of oligonucleotide tag sequences (e.g., Oligopaints) that is specific for a set of binding patterns in a genome (e.g., a human genome). In some embodiments of any of the aspects, an array or palette is specific for a set of chromosomal aberrations (e.g., one or more of a translocation, an insertion, an inversion, a deletion, a duplication, a transposition, aneuploidy, polyploidy, complex rearrangement and telomere loss) associated with one or more disorders described herein. In some embodiments of any of the aspects, the kits described herein are particularly suited for diagnostic and/or prognostic use for detecting one or more disorders described herein in clinical settings (e.g., hospitals, medical clinics, medical offices, diagnostic laboratories, research laboratories and the like (e.g., for patient diagnosis and/or prognosis, prenatal diagnosis and/or prognosis and the like).

In some embodiments of any of the aspects, a kit provides instructions for amplifying the plurality of specific oligonucleotide tag sequences (e.g., Oligopaints) provided in the kit. In some embodiments of any of the aspects, the kit provides instructions for detectably and/or retrievably labeling one or more target nucleic acid sequences (e.g., one or more chromosomes or sub-chromosomal regions) using the amplified oligonucleotide tags (e.g., an Oligopaint). In some embodiments of any of the aspects, the kit provides instructions for detectably and/or retrievably labeling one or more target nucleic acid sequences (e.g., one or more chromosomes or sub-chromosomal regions) using the oligonucleotide tags (e.g., an Oligopaint) and readout molecules. In some embodiments of any of the aspects, a kit provides instructions for effectively removing one or more of the plurality of specific oligonucleotide tag sequences (e.g., Oligopaints) during the amplification step by including one or more unlabeled amplification primers that hybridizes to the one or more oligonucleotide sequences that one wishes to remove, such that the one or more target nucleic acid sequences is rendered not detectably and/or retrievably labeled.

In some embodiments of any of the aspects, systems and methods described herein may be implemented with any type of hardware and/or software, and may be a pre-programmed general purpose computing device. For example, the system may be implemented using a server, a personal computer, a portable computer, a thin client, or any suitable device or devices. The disclosure and/or components thereof may be a single device at a single location, or multiple devices at a single, or multiple, locations that are connected together using any appropriate communication protocols over any communication medium such as electric cable, fiber optic cable, or in a wireless manner.

It should also be noted that the disclosure is illustrated and discussed herein as having a plurality of modules which perform particular functions. It should be understood that these modules are merely schematically illustrated based on their function for clarity purposes only, and do not necessary represent specific hardware or software. In this regard, these modules may be hardware and/or software implemented to substantially perform the particular functions discussed. Moreover, the modules may be combined together within the disclosure, or divided into additional modules based on the particular function desired. Thus, the disclosure should not be construed to limit the present invention, but merely be understood to illustrate one example implementation thereof.

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Implementations of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer to-peer networks).

Implementations of the subject matter and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate physical components or media (e.g., multiple CDs, disks, or other storage devices).

The operations described in this specification can be implemented as operations performed by a “data processing apparatus” on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

As used herein, the term “chromosome” refers to the support for the genes carrying heredity in a living cell, including DNA, protein, RNA and other associated factors. The conventional international system for identifying and numbering the chromosomes of the human genome is used herein. The size of an individual chromosome may vary within a multi-chromosomal genome and from one genome to another. A chromosome can be obtained from any species. A chromosome can be obtained from an adult subject, a juvenile subject, an infant subject, from an unborn subject (e.g., from a fetus, e.g., via prenatal test such as amniocentesis, chorionic villus sampling, and the like or directly from the fetus, e.g., during a fetal surgery) from a biological sample (e.g., a biological tissue, fluid or cells (e.g., sputum, blood, blood cells, tissue or fine needle biopsy samples, urine, cerebrospinal fluid, peritoneal fluid, and pleural fluid, or cells therefrom) or from a cell culture sample (e.g., primary cells, immortalized cells, partially immortalized cells or the like). In certain exemplary embodiments, one or more chromosomes can be obtained from one or more genera including, but not limited to, Homo, Drosophila, Caenorhabiditis, Danio, Cyprinus, Equus, Canis, Ovis, Ocorynchus, Salmo, Bos, Sus, Gallus, Solanum, Triticum, Oryza, Zea, Hordeum, Musa, Avena, Populus, Brassica, Saccharum and the like.

As used herein, the term “chromosome banding” refers to differential staining of chromosomes resulting in a pattern of transverse bands of distinguishable (e.g., differently or alternately colored) regions, that is characteristic for the individual chromosome or chromosome region (i.e., the “banding pattern”). Conventional banding techniques include G-banding (Giemsa stain), Q-banding (Quinacrine mustard stain), R-banding (reverse-Giemsa), and C-banding (centromere banding).

As used herein, the term “karyotype” refers to the chromosome characteristics of an individual cell, cell line or genome of a given species, as defined by both the number and morphology of the chromosomes. Karyotype can refer to a variety of chromosomal rearrangements including, but not limited to, translocations, insertional translocations, inversions, deletions, duplications, transpositions, anueploidies, complex rearrangements, telomere loss and the like. Typically, the karyotype is presented as a systematized array of prophase or metaphase (or otherwise condensed) chromosomes from a photomicrograph or computer-generated image. Interphase chromosomes may also be examined.

As used herein, the terms “chromosomal aberration” or “chromosome abnormality” refer to a deviation between the structure of the subject chromosome or karyotype and a normal (i.e., non-aberrant) homologous chromosome or karyotype. The deviation may be of a single base pair or of many base pairs. The terms “normal” or “non-aberrant,” when referring to chromosomes or karyotypes, refer to the karyotype or banding pattern found in healthy individuals of a particular species and gender. Chromosome abnormalities can be numerical or structural in nature, and include, but are not limited to, aneuploidy, polyploidy, inversion, translocation, deletion, duplication and the like. Chromosome abnormalities may be correlated with the presence of a pathological condition or with a predisposition to developing a pathological condition. Chromosome aberrations and/or abnormalities can also refer to changes that are not associated with a disease, disorder and/or a phenotypic change. Such aberrations and/or abnormalities can be rare or present at a low frequency (e.g., a few percent of the population (e.g., polymorphic)).

Disorders associated with one or more chromosome abnormalities include, but are not limited to: autosomal abnormalities (e.g., trisomies (Down syndrome (chromosome 21), Edwards syndrome (chromosome 18), Patau syndrome (chromosome 13), trisomy 9, Warkany syndrome (chromosome 8), trisomy 22/cat eye syndrome, trisomy 16); monosomies and/or deletions (Wolf-Hirschhorn syndrome (chromosome 4), Cri du chat/Chromosome 5q deletion syndrome (chromosome 5), Williams syndrome (chromosome 7), Jacobsen syndrome (chromosome 11), Miller-Dieker syndrome/Smith-Magenis syndrome (chromosome 17), Di George's syndrome (chromosome 22), genomic imprinting (Angelman syndrome/Prader-Willi syndrome (chromosome 15))); X/Y-linked abnormalities (e.g., monosomies (Turner syndrome (XO), trisomy or tetrasomy and/or other karyotypes or mosaics (Klinefelter's syndrome (47 (XXY)), 48 (XXYY), 48 (XXXY), 49 (XXXYY), 49 (XXXXY), Triple X syndrome (47 (XXX)), 48 (XXXX), 49 (XXXXX), 47 (XYY), 48 (XYYY), 49 (XYYYY), 46 (XX/XY)); translocations (e.g., leukemia or lymphoma (e.g., lymphoid (e.g., Burkitt's lymphoma t(8 MYC; 14 IGH), follicular lymphoma t(14 IGH; 18 BCL2), mantle cell lymphoma/multiple myeloma t(11 CCND1; 14 IGH), anaplastic large cell lymphoma t(2 ALK; 5 NPM1), acute lymphoblastic leukemia) or myeloid (e.g., Philadelphia chromosome t(9 ABL; 22 BCR), acute myeloblastic leukemia with maturation t(8 RUNX1T1; 21 RUNX1), acute promyelocytic leukemia t(15 PML, 17 RARA), acute megakaryoblastic leukemia t(1 RBM15; 22 MKL1))) or other (e.g., Ewing's sarcoma t(11 FiI; 22 EWS), synovial sarcoma t(x SYT; 18 SSX), dermatofibrosarcoma protuberans t(17 COL1A1; 22 PDGFB), myxoid liposarcoma t(12 DDIT3; 16 FUS), desmoplastic small round cell tumor t(11 WT1; 22 EWS), alveolar rhabdomyosarcoma t(2 PAX3; 13 FOXO1) t (1 PAX7; 13 FOXO1))); gonadal dysgenesis (e.g., mixed gonadal dysgenesis, XX gonadal dysgenesis); and other abnormalities (e.g., fragile X syndrome, uniparental disomy). Disorders associated with one or more chromosome abnormalities also include, but are not limited to, Beckwith-Wiedmann syndrome, branchio-oto-renal syndrome, Cri-du-Chat syndrome, De Lange syndrome, holoprosencephaly, Rubinstein-Taybi syndrome and WAGR syndrome.

Disorders associated with one or more chromosome abnormalities also include cellular proliferative disorders (e.g., cancer). As used herein, the term “cellular proliferative disorder” includes disorders characterized by undesirable or inappropriate proliferation of one or more subset(s) of cells in a multicellular organism. The term “cancer” refers to various types of malignant neoplasms, most of which can invade surrounding tissues, and may metastasize to different sites (see, for example, PDR Medical Dictionary 1st edition, 1995). The terms “neoplasm” and “tumor” refer to an abnormal tissue that grows by cellular proliferation more rapidly than normal and continues to grow after the stimuli that initiated proliferation is removed (see, for example, PDR Medical Dictionary 1st edition, 1995). Such abnormal tissue shows partial or complete lack of structural organization and functional coordination with the normal tissue which may be either benign (i.e., benign tumor) or malignant (i.e., malignant tumor).

Disorders associated with one or more chromosome abnormalities also include brain disorders including, but not limited to, acoustic neuroma, acquired brain injury, Alzheimer's disease, amyotrophic lateral diseases, aneurism, aphasia, arteriovenous malformation, attention deficit hyperactivity disorder, autism Batten disease, Bechet's disease, blepharospasm, brain tumor, cerebral palsy Charcot-Marie-Tooth disease, chiari malformation, CIDP, non-Alzheimer-type dementia, dysautonomia, dyslexia, dysprazia, dystonia, epilepsy, essential tremor, Friedrich's ataxia, gaucher disease, Gullian-Barre syndrome, headache, migraine, Huntington's disease, hydrocephalus, Meniere's disease, motor neuron disease, multiple sclerosis, muscular dystrophy, myasthenia gravis, narcolepsy, Parkinson's disease, peripheral neuropathy, progressive supranuclear palsy, restless legs syndrome, Rett syndrome, schizophrenia, Shy Drager syndrome, stroke, subarachnoid hemorrhage, Sydenham's syndrome, Tay-Sachs disease, Tourette syndrome, transient ischemic attack, transverse myelitis, trigeminal neuralgia, tuberous sclerosis and von Hippel-Lindau syndrome.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99% , or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

In some embodiments of any of the aspects, the reference sample or level is the sample or level of the sample itself prior to being contacted with a composition described herein. In some embodiments of any of the aspects, the reference sample or level is the sample or level of a composition described herein prior to being contacted with the sample. In some embodiments of any of the aspects, the reference can be a sample contacted with compositions not comprising detection molecules. In some embodiments of any of the aspects, the reference can be a sample contacted with compositions comprising recognition domains that are not specific to the sample. In some embodiments of any of the aspects, the reference can also be a level obtained from a control sample, a pooled sample of control individuals, or a numeric value or range of values based on the same.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g. activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, the polypeptide described herein (or a nucleic acid encoding such a polypeptide) can be a functional fragment of one of the amino acid sequences described herein. As used herein, a “functional fragment” is a fragment or segment of a peptide which retains at least 50% of the wildtype reference polypeptide's activity according to the assays described below herein. A functional fragment can comprise conservative substitutions of the sequences disclosed herein.

In some embodiments, the polypeptide described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan.

A variant amino acid or DNA sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double- stranded DNA. Alternatively, it can be a single-stranded nucleic acid not derived from any double-stranded DNA. In some embodiments of any of the aspects, a single-stranded nucleic acid is produced by in-vitro transcription followed by reverse transcription. In some embodiments of any of the aspects, a single-stranded nucleic acid is produced by exposure to nicking endonuclease. In some embodiments of any of the aspects, a single-stranded nucleic acid is synthesized de novo. In one aspect, the nucleic acid can be DNA. In another aspect, the nucleic acid can be RNA. Suitable DNA can include, e.g., genomic DNA or cDNA. Suitable RNA can include, e.g., mRNA.

The term “expression” refers to the cellular processes involved in producing RNA and proteins and as appropriate, secreting proteins, including where applicable, but not limited to, for example, transcription, transcript processing, translation and protein folding, modification and processing. Expression can refer to the transcription and stable accumulation of sense (mRNA) or antisense RNA derived from a nucleic acid fragment or fragments of the invention and/or to the translation of mRNA into a polypeptide.

In some embodiments, the expression of a biomarker(s), target(s), or gene/polypeptide described herein is/are tissue-specific. In some embodiments, the expression of a biomarker(s), target(s), or gene/polypeptide described herein is/are global. In some embodiments, the expression of a biomarker(s), target(s), or gene/polypeptide described herein is systemic.

“Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

In some embodiments, the methods described herein relate to measuring, detecting, or determining the level of at least one target molecule. As used herein, the term “detecting” or “measuring” refers to observing a signal from, e.g. a probe, label, or target molecule to indicate the presence of an analyte in a sample. Any method known in the art for detecting a particular label moiety can be used for detection. Exemplary detection methods include, but are not limited to, spectroscopic, fluorescent, photochemical, biochemical, immunochemical, electrical, optical or chemical methods. In some embodiments of any of the aspects, measuring can be a quantitative observation.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

In some embodiments of any of the aspects, the nucleic acid (e.g., oligonucleotide tag, e.g., an Oligopaint) described herein is exogenous. In some embodiments of any of the aspects, the nucleic acid (e.g., oligonucleotide tag, e.g., an Oligopaint) described herein is ectopic. In some embodiments of any of the aspects, the nucleic acid (e.g., oligonucleotide tag, e.g., an Oligopaint) described herein is not endogenous.

The term “exogenous” refers to a substance present in a cell other than its native source. The term “exogenous” when used herein can refer to a nucleic acid (e.g. a nucleic acid encoding a polypeptide) or a polypeptide that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell. As used herein, “ectopic” refers to a substance that is found in an unusual location and/or amount. An ectopic substance can be one that is normally found in a given cell, but at a much lower amount and/or at a different time. Ectopic also includes substance, such as a polypeptide or nucleic acid that is not naturally found or expressed in a given cell in its natural environment.

As used herein, “contacting” refers to any suitable means for delivering, or exposing, an agent to at least one cell. Exemplary delivery methods include, but are not limited to, direct delivery to cell culture medium, perfusion, injection, or other delivery method well known to one skilled in the art. In some embodiments, contacting comprises physical human activity, e.g., an injection; an act of dispensing, mixing, and/or decanting; and/or manipulation of a delivery device or machine.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean ±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used herein, the term “corresponding to” refers to an amino acid or nucleotide at the enumerated position in a first polypeptide or nucleic acid, or an amino acid or nucleotide that is equivalent to an enumerated amino acid or nucleotide in a second polypeptide or nucleic acid. Equivalent enumerated amino acids or nucleotides can be determined by alignment of candidate sequences using degree of homology programs known in the art, e.g., BLAST.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:
1. A method of analyzing at least one target molecule in a sample, the method comprising:
   a. contacting the sample with at least one oligonucleotide tag, each oligonucleotide tag comprising:
      i. a recognition domain that binds specifically to a target molecule to be analyzed, and
      ii. at least one street comprising at least one cassette, each cassette comprising:
         1. a barcode region comprising at least 1 nucleotide, flanked on at least one side by an anchor region;
   wherein each oligonucleotide tag's street is unique from the streets of the other oligonucleotide tags of step (a) at least in that A. the spatial order of the cassettes within the street differs or B. that the sequence of the barcode region differs from the barcode regions of the other oligonucleotide tags of step (a);
   b. contacting the sample with at least two readout molecules, wherein each readout molecule comprises:
      i. an oligonucleotide that hybridizes specifically with a cassette of at least one oligonucleotide tag used in step (a); and
      ii. a detection molecule;
   wherein the at least two readout molecules collectively comprise at least two distinguishable detection molecules; and
c. detecting the relative spatial order of the detection molecules hybridized to at least one oligonucleotide tag, wherein the at least one oligonucleotide tag is hybridized to the at least one target molecule, whereby the relative spatial order of the detection molecules permits identification of which oligonucleotide tag is hybridized to the target molecule at that location.
2. The method of any of the preceding paragraphs, wherein the barcode region comprises 1-10 nucleotides.
3. The method of any of the preceding paragraphs, wherein the street comprises at least 3 cassettes.
4. The method of any of the preceding paragraphs, wherein the barcode region is flanked on each side by an anchor region.
5. The method of any of the preceding paragraphs, wherein the anchor regions of all of the oligonucleotide tags are constant.
6. The method of any of the preceding paragraphs, wherein the specific hybridization of a readout molecule to a cassette is determined by the identity of the barcode region.
7. The method of any of the preceding paragraphs, wherein the detection molecule is a fluorophore.
8. The method of any of the preceding paragraphs, wherein the detecting is performed with fluorescence microscopy.
9. The method of any of the preceding paragraphs, wherein the detection molecule comprises biotin, amines, metals, anchoring molecules, or acrydite.
10. The method of any of the preceding paragraphs, wherein the detecting is performed with at least single cell resolution.
11. The method of any of the preceding paragraphs, wherein step (b) comprises contacting the sample with at least 4 readout molecules.
12. The method of any of the preceding paragraphs, wherein step (b) comprises contacting the sample with a group of readout molecules that collectively comprise at least 3 distinguishable detection molecules.
13. The method of any of the preceding paragraphs, wherein step (b) comprises contacting the sample with a group of readout molecules that collectively comprise at least 4 distinguishable detection molecules.
14. The method of any of the preceding paragraphs, wherein at least 2 target molecules are analyzed concurrently.
15. The method of any of the preceding paragraphs, wherein at least 3 target molecules are analyzed concurrently.
16. The method of any of the preceding paragraphs, wherein at least 10 target molecules are analyzed concurrently.
17. The method of any of the preceding paragraphs, wherein at least 20 target molecules are analyzed concurrently.
18. The method of any of the preceding paragraphs, wherein the target molecule is a nucleic acid, a polypeptide, a cell surface molecule, or an inorganic material.
19. The method of any of the preceding paragraphs, wherein the target molecule is a DNA or mRNA.
20. The method of any of the preceding paragraphs, wherein the sample is a cell, cell culture, or tissue sample.
21. A system for analyzing at least one target molecule in a sample, the system comprising:
   a. a detector that can detect at least two detectable molecules;
   b. at least one oligonucleotide tag, each oligonucleotide tag comprising:
      i. a recognition domain that binds specifically to a target molecule to be analyzed, and
      ii. a street comprising at least one cassette, each cassette comprising:
         1. a barcode region comprising at least 1 nucleotide, flanked on at least one side by an anchor region;
   wherein each oligonucleotide tag's street is unique from the streets of the other oligonucleotide tags of (b) at least in that A. the spatial order of the cassettes within the street differs or B. that the sequence of the barcode region differs from the barcode regions of the other oligonucleotide tags of (b); and
   c. at least two readout molecules, wherein each readout molecule comprises:
      i. an oligonucleotide that hybridizes specifically with a cassette of at least one oligonucleotide tag used in (b); and
      ii. a detection molecule;
   wherein the at least two readout molecules collectively comprise at least two distinguishable detection molecules; and
   wherein a sample is contacted with the at least one oligonucleotide tag and the at least two readout molecules, and the relative spatial order of the detection molecules hybridized to at least one oligonucleotide tag is detected, wherein the at least one oligonucleotide tag is hybridized to the at least one target molecule, whereby the relative spatial order of the detection molecules permits identification of which oligonucleotide tag is hybridized to the target molecule at that location.

### EXAMPLES

### Example 1

Compared to existing technologies, the methods and compositions described herein permit the analysis of DNA, mRNAs, proteins, etc. using microscopy at the single cell level, by utilizing a multiplexing technique, OligoCASSEQ, to visualize and identify thousands of targets in the same cell, delivering a more global and complete view of cellular processes. Unlike current microscopy technologies that are limited to analyzing 4-5 targets at once, OligoCASSEQ achieves high levels of multiplexing in an efficient and cost effective manner. First, OligoCASSEQ does not use enzymes, which can be costly and inefficient. Secondly, OligoCASSEQ does not require lengthy oligos, which decrease accuracy while increasing costs.

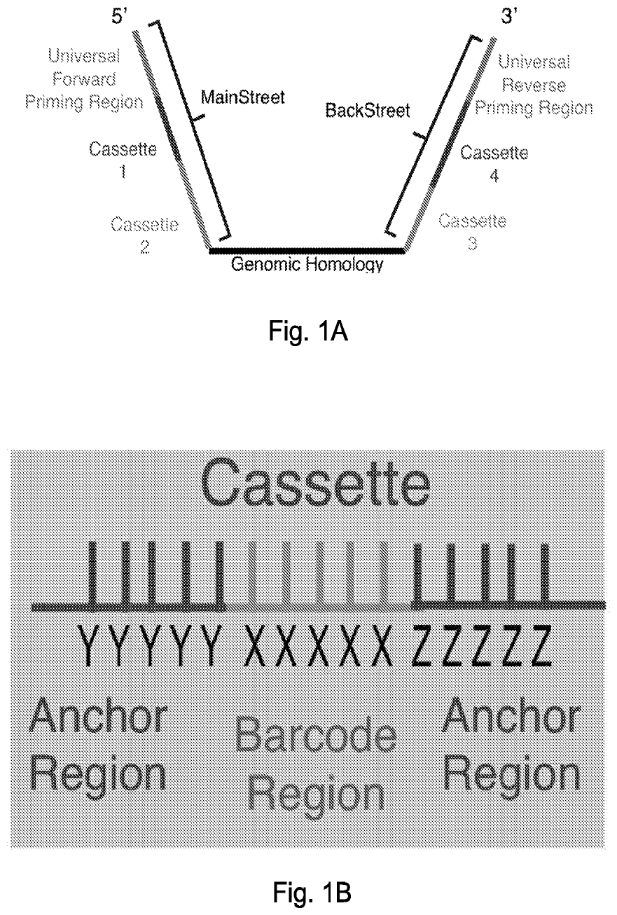

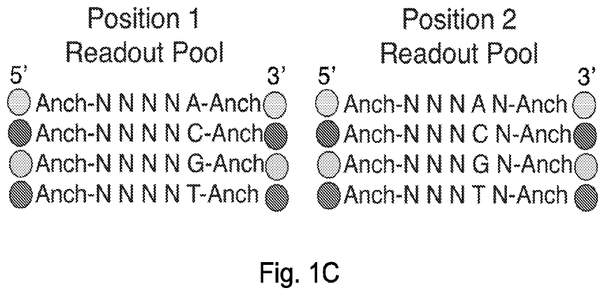

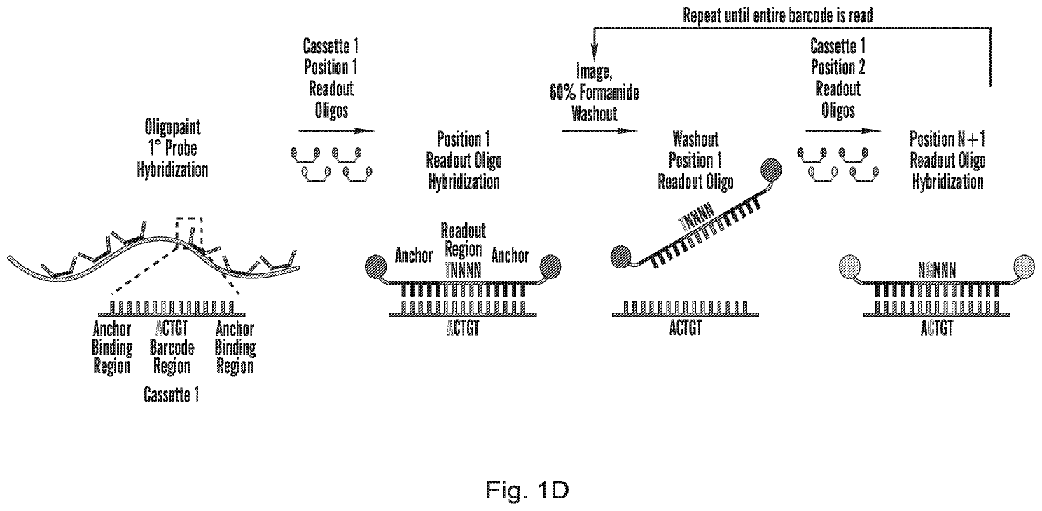

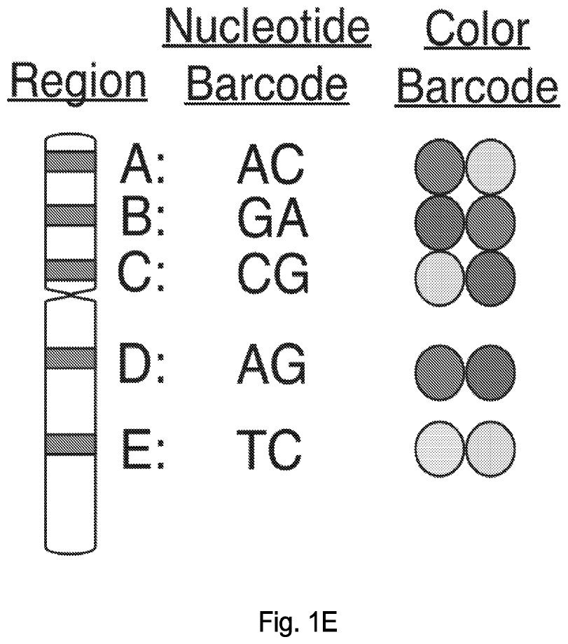

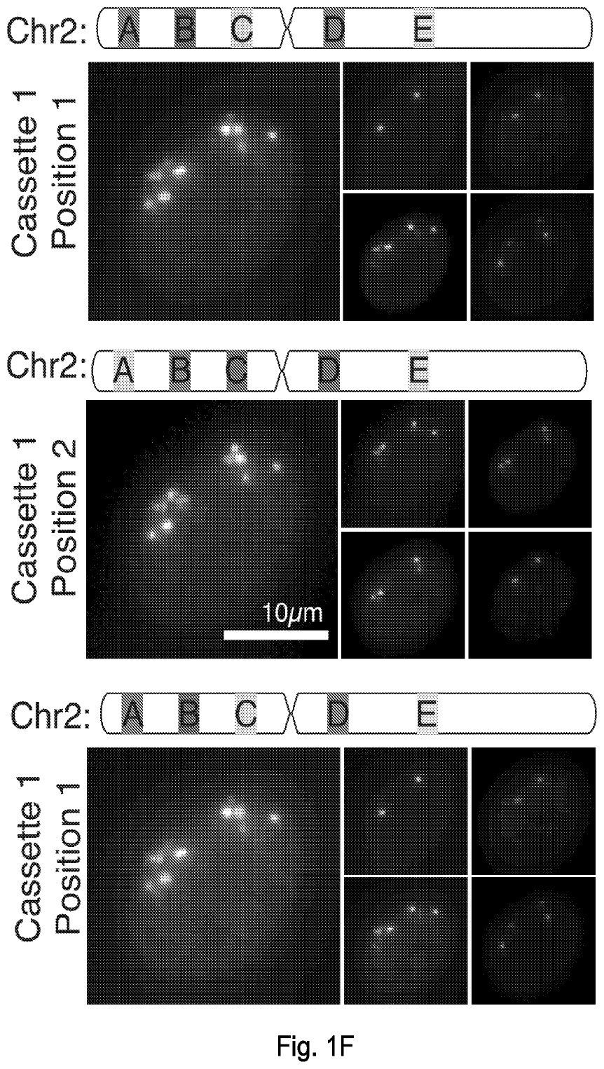

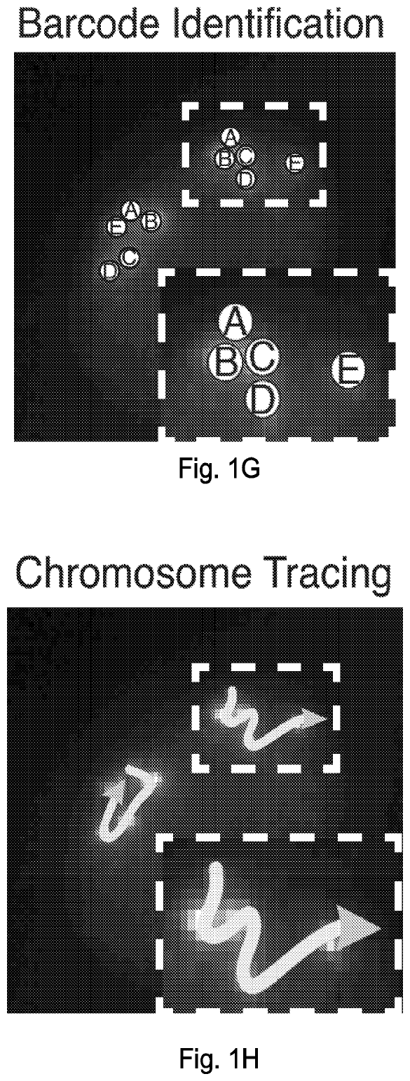

The technology described herein, referred to at times as “OligoCASSEQ”, is a technology that allows for highly multiplexed target identification at the single cell level. In this exemplary embodiment, DNA is targeted by Oligopaints with “streets” containing “cassettes” (see e.g., FIG. 1A). Cassettes consist of a variable barcode region flanked by constant anchor regions on each side (see e.g., FIG. 1B). Barcodes are sequenced using fluor labeled oligos (e.g., Readouts, also referred to as readout molecules) specific to a nucleotide at each barcode position (see e.g., FIG. 1C). Readouts recognize and competitively bind cassettes via anchor regions. Complementary binding is dictated by Readout recognition of specific barcode sequences (see e.g., FIG. 1D). This design allows for the cassette to be compact and for the reduction in the amount of readouts required. A five nucleotide barcode can distinguish 1024 targets (4⁵).

OligoCASSEQ is complementary to Oligopaint technology. That is to say that OligoCASSEQ can work to multiplex/decode anything with an oligonucleotide tag on it, which can include but is not limited to oligonucleotide-tagged oligos (e.g., Oligopaint or any other DNA-binding entity), oligonucleotide-tagged antibodies (e.g., nanobodies), oligonucleotide-tagged small molecules (e.g., for the purpose of drug screens), oligonucleotide-tagged cells, and non-biological materials (metals, chemicals, etc.) with oligonucleotide tags. DNA targets are hybridized with Oligopaints. The key difference is that with OligoCASSEQ, nucleotide cassette sequences are encoded into the Oligopaint non-genomic targeting “Streets”. These cassettes allow for barcoding of Oligopaints and thus, high levels of multiplexing.

As described herein, the target can be DNA. OligoCASSEQ is also amenable to other targets as well (e.g., RNA, protein, etc.). OligoCASSEQ allows for the identification of vast numbers of targets via microscopy. These targets include, but are not limited to: DNA, RNA, proteins, cells, inorganic materials.

Demonstrated herein is the use of OligoCASSEQ to trace chromosomes in-situ. As a non-limiting example, OligoCASSEQ can be used to determine the localization of five loci along human Chromosome 2 (Chr.2; see e.g., FIG. 1E-FIG. 1H). A two-nucleotide 4-color barcode was used (see e.g., FIG. 1E). Micrograph images of PGP1-F cells show OligoCASSEQ interrogation of two barcode positions (see e.g., the top and middle sections), followed by re-interrogation of the 1st position in the same nucleus (see e.g., the bottom section). A schematic above each micrograph displays the color code of specific loci at different barcode positions (see e.g., FIG. 1F). The readouts on the barcodes were identified and then decoded (see e.g., FIG. 1G). The spatial location of each chromosome was then traced (see e.g., FIG. 1H).

OligoCASSEQ addresses the need to target and identify multiple (>5) targets at once. Only 4-5 targets can currently be studied at a time, due to microscopes being able to distinguish only 4-5 colors at a time. OligoCASSEQ solves this problem through barcoding and multiplexing. Instead of a target being represented by 1 color, the target is represented by a sequence of 4 colors, exponentially increasing the number of targets one can interrogate.

OligoCASSEQ is superior to alternatives due to 1) no requirement of enzymes, 2) reduction in length of oligos, and 3) reduction in complexity and number of readout oligos required.

1. A method of analyzing at least one target molecule in a sample, the method comprising:
(a) contacting the sample with at least one oligonucleotide tag, each oligonucleotide tag comprising:
(i) a recognition domain that binds specifically to a target molecule to be analyzed, and
(ii) at least one street comprising at least one cassette, each cassette comprising:
(1) a barcode region comprising at least 1 nucleotide, flanked on at least one side by an anchor region;
wherein each oligonucleotide tag's street is unique from the streets of the other oligonucleotide tags of step (a) at least in that (A) the spatial order of the cassettes within the street differs or (B) that the sequence of the barcode region differs from the barcode regions of the other oligonucleotide tags of step (a);
(b) contacting the sample with at least two readout molecules, wherein each readout molecule comprises:
(i) an oligonucleotide that hybridizes specifically with a cassette of at least one oligonucleotide tag used in step (a); and
(ii) a detection molecule;
wherein the at least two readout molecules collectively comprise at least two distinguishable detection molecules; and
(c) detecting the relative spatial order of the detection molecules hybridized to at least one oligonucleotide tag, wherein the at least one oligonucleotide tag is hybridized to the at least one target molecule, whereby the relative spatial order of the detection molecules permits identification of which oligonucleotide tag is hybridized to the target molecule at that location.

2. The method of claim 1, wherein the barcode region comprises 1-10 nucleotides.

3. The method of claim 1, wherein the street comprises at least 3 cassettes.

4. The method of claim 1, wherein the barcode region is flanked on each side by an anchor region.

5. The method of claim 1, wherein the anchor regions of all of the oligonucleotide tags are constant.

6. The method of claim 1, wherein the specific hybridization of a readout molecule to a cassette is determined by the identity of the barcode region.

7. The method of claim 1, wherein the detection molecule is a fluorophore.

8. The method of claim 7, wherein the detecting is performed with fluorescence microscopy.

9. The method of claim 1, wherein the detection molecule comprises biotin, amines, metals, anchoring molecules, or acrydite.

10. The method of claim 1, wherein the detecting is performed with at least single cell resolution.

11. The method of claim 1, wherein step (b) comprises contacting the sample with at least 4 readout molecules.

12. The method of claim 1, wherein step (b) comprises contacting the sample with a group of readout molecules that collectively comprise at least 3 distinguishable detection molecules.

13. The method of claim 1, wherein step (b) comprises contacting the sample with a group of readout molecules that collectively comprise at least 4 distinguishable detection molecules.

14. The method of claim 1, wherein at least 2 target molecules are analyzed concurrently.

15. The method of claim 1, wherein at least 3 target molecules are analyzed concurrently.

16. The method of any claim 1, wherein at least 10 target molecules are analyzed concurrently.

17. (canceled)

18. The method of claim 1, wherein the target molecule is a nucleic acid, a polypeptide, a cell surface molecule, or an inorganic material.

19. The method of claim 1, wherein the target molecule is a DNA or mRNA.

20. The method of claim 1, wherein the sample is a cell, cell culture, or tissue sample.

21. A system for analyzing at least one target molecule in a sample, the system comprising:
(a) a detector that can detect at least two detectable molecules;
(b) at least one oligonucleotide tag, each oligonucleotide tag comprising:
(i) a recognition domain that binds specifically to a target molecule to be analyzed, and
(ii) a street comprising at least one cassette, each cassette comprising:
(1) a barcode region comprising at least 1 nucleotide, flanked on at least one side by an anchor region;
wherein each oligonucleotide tag's street is unique from the streets of the other oligonucleotide tags of (b) at least in that (A) the spatial order of the cassettes within the street differs or (B) that the sequence of the barcode region differs from the barcode regions of the other oligonucleotide tags of (b); and
(c) at least two readout molecules, wherein each readout molecule comprises:
(i) an oligonucleotide that hybridizes specifically with a cassette of at least one oligonucleotide tag used in (b); and
(ii) a detection molecule;
wherein the at least two readout molecules collectively comprise at least two distinguishable detection molecules; and
wherein a sample is contacted with the at least one oligonucleotide tag and the at least two readout molecules, and the relative spatial order of the detection molecules hybridized to at least one oligonucleotide tag is detected, wherein the at least one oligonucleotide tag is hybridized to the at least one target molecule, whereby the relative spatial order of the detection molecules permits identification of which oligonucleotide tag is hybridized to the target molecule at that location.


SEQUENCE LISTING (4 sequences)

SEQ ID NO: 1 - DNA, 5 bp, Artificial Sequence
  synthetic oligonucleotide
gtctc

SEQ ID NO: 2 - DNA, 5 bp, Artificial Sequence
  synthetic oligonucleotide
cacta

SEQ ID NO: 3 - DNA, 5 bp, Artificial Sequence
  synthetic oligonucleotide
gcccg

SEQ ID NO: 4 - DNA, 5 bp, Artificial Sequence
  synthetic oligonucleotide
tgtgc